(19) United States
(12) Patent Application Publication
(10) Pub. No.: US 2024/0271162 A1
(43) Pub. Date: Aug. 15, 2024

(54) RNA POLYMERASE III PROMOTERS AND METHODS OF USE

(71) Applicant: LifeEDIT Therapeutics, Inc., Durham, NC (US)

(72) Inventors: Tyson D. Bowen, Morrisville, NC (US); Alexandra Briner Crawley, Cary, NC (US); Tedd D. Elich, Durham, NC (US)

(73) Assignee: LifeEDIT Therapeutics, Inc., Durham, NC (US)

(21) Appl. No.: 18/568,638

(22) PCT Filed: Jun. 10, 2022
(86) PCT No.: PCT/US2022/032940

§ 371 Date: Dec. 8, 2023

Related U.S. Application Data
(60) Provisional application No. 63/209,660, filed on Jun. 11, 2021.

(51) Int. Cl. C12N 15/90; C12N 9/22; C12N 9/78; C12N 15/11

(52) U.S. Cl. CPC C12N 15/90; C12N 9/22; C12N 9/78; C12N 15/11; C12Y 305/04; C12N 2310/20

(57) ABSTRACT

Compositions and methods useful for expressing an RNA are provided. Compositions comprise nucleic acid molecules comprising an RNA polymerase III promoter and expression constructs comprising the novel promoters operably linked to a coding sequence. The promoters can initiate transcription of RNA-coding nucleotide sequences through the recruitment of RNA polymerase III (pol III) or, in some embodiments, polypeptide-encoding nucleotide sequences through the recruitment of RNA polymerase II. The promoters thus find use in transcribing nucleotide sequences, including but not limited to, those encoding guide RNAs, and polypeptide-encoding sequences. Such expression constructs comprising the novel promoters operably linked to nucleotide sequences encoding guide RNAs find use in binding and/or modifying the sequence of a target nucleic acid molecule, modifying the expression of a target gene, or detecting a target nucleotide sequence in cells comprising an RNA-guided nuclease (RGN) or RNA-guided nucleotide-binding polypeptide (RGNBP).

### CROSS-REFERENCE TO RELATED APPLICATION

This application claims priority to U.S. Provisional Patent Application No. 63/209,660, filed Jun. 11, 2021, which is fully incorporated by reference herein.

### REFERENCE TO A SEQUENCE LISTING SUBMITTED ELECTRONICALLY AS A TEXT FILE

The instant application contains a Sequence Listing which has been submitted in ASCII format via EFS-Web and is hereby incorporated by reference in its entirety. Said ASCII copy, created on Jun. 9, 2022, is named L103438_1250WO_0149_9_SL.txt, and is 714 KB in size.

### FIELD OF THE INVENTION

The present invention relates to the field of molecular biology and gene editing.

### BACKGROUND OF THE INVENTION

Small RNAs are used in many genome editing or expression modulation techniques, including gene silencing through the RNA interference (RNAi) pathway. Genome editing techniques utilizing RNA-guided nucleases, such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than previous methods of genome editing that required the generation of chimeric nucleases for each target sequence. Small RNAs useful in genome editing or gene silencing are typically expressed from RNA polymerase III promoters, rather than RNA polymerase II-regulated promoters which are more generally employed for the expression of polypeptides. In some instances, however, pol III promoters can also recruit RNA polymerase II and can initiate transcription of an operably linked polypeptide-encoding sequence.

### BRIEF SUMMARY OF THE INVENTION

Compositions and methods for expressing an RNA and preparing an RNA expression construct are provided. Compositions comprise nucleic acid molecules comprising an RNA polymerase III promoter and expression constructs comprising the novel promoters operably linked to a coding sequence. The promoters can initiate transcription of RNA-coding nucleotide sequences through the recruitment of RNA polymerase III (pol III) or, in some embodiments, polypeptide-encoding nucleotide sequences through the recruitment of RNA polymerase II. The promoters thus find use in transcribing nucleotide sequences, including but not limited to, those encoding guide RNAs, and polypeptide-encoding sequences. Such expression constructs comprising the novel promoters operably linked to nucleotide sequences encoding guide RNAs find use in binding and/or modifying the sequence of a target nucleic acid molecule, modifying the expression of a target gene, or detecting a target nucleic acid molecule in cells comprising an RNA-guided nuclease (RGN) or RNA-guided nucleotide-binding polypeptide (RGNBP). Also provided are polynucleotides comprising the expression cassettes comprising the promoters operably linked to a guide RNA-encoding nucleotide sequence and RGN-encoding nucleotide sequences.

In one aspect, the present disclosure provides a promoter comprising a nucleotide sequence having at least 90% sequence identity to the nucleotide sequence set forth as any one of SEQ ID NOs: 6-10.

In some embodiments of the above aspect, the promoter comprises a nucleotide sequence having at least 95% sequence identity to the nucleotide sequence set forth as any one of SEQ ID NOs: 6-10. In some embodiments of the above aspect, the promoter comprises a nucleotide sequence having at least 96%, 97%, 98%, or at least 99% sequence identity to the nucleotide sequence set forth as any one of SEQ ID NOs: 6-10. In some embodiments, the promoter comprises a nucleotide sequence having 100% sequence identity to the nucleotide sequence set forth as any one of SEQ ID NOs: 6-10.

In some embodiments of the above aspect, the promoter comprises a TATA box sequence. In some embodiments, the promoter comprises an octamer (OCT) sequence. In some embodiments, the promoter comprises a SPH sequence. In some embodiments, the promoter comprises a proximal sequence element (PSE).

In another aspect, the present disclosure provides a vector comprising the promoter as described hereinabove.

In some embodiments of the above aspect, the vector is a viral vector. In some embodiments, the viral vector is an adeno-associated viral (AAV) vector.

In another aspect, the present disclosure provides an expression construct comprising the promoter as described hereinabove and a coding sequence, wherein the coding sequence is operably linked to the promoter.

In some embodiments of the above aspect, the coding sequence is heterologous to the promoter.

In some embodiments of the above aspect, the coding sequence encodes an RNA. In some embodiments of the above aspect, the RNA comprises a guide RNA.

In some embodiments of the above aspect, the coding sequence encodes a polypeptide.

In another aspect, the present disclosure provides a polynucleotide comprising the expression construct as described hereinabove and an RNA-guided nuclease (RGN)-encoding nucleotide sequence, wherein the RGN is capable of binding the guide RNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA is a crRNA.

In another aspect, the present disclosure provides a polynucleotide comprising the expression construct as described hereinabove and a tracrRNA-encoding nucleotide sequence.

In some embodiments of the above aspect, the polynucleotide further comprises an RNA polymerase III (pol III) promoter operably linked to the tracrRNA-encoding nucleotide sequence.

In some embodiments of the above aspect, the pol III promoter comprises a nucleotide sequence having at least 90% sequence identity to the nucleotide sequence set forth as any one of SEQ ID NOs: 1-10. In some embodiments, the pol III promoter comprises a nucleotide sequence having at least 95% sequence identity to the nucleotide sequence set forth as any one of SEQ ID NOs: 1-10. In some embodiments, the pol III promoter comprises a nucleotide sequence having at least 96%, 97%, 98%, or at least 99% sequence identity to the nucleotide sequence set forth as any one of SEQ ID NOs: 1-10. In some embodiments, the pol III promoter comprises a nucleotide sequence having 100% sequence identity to the nucleotide sequence set forth as any one of SEQ ID NOs: 1-10.

In some embodiments of the above aspect, the polynucleotide further comprises an RNA-guided nuclease (RGN)-encoding nucleotide sequence, wherein the RGN is capable of binding the crRNA and the tracrRNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA is a tracrRNA.

In another aspect, the present disclosure provides a polynucleotide comprising the expression construct as described hereinabove and a crRNA-encoding nucleotide sequence.

In some embodiments of the above aspect, the polynucleotide further comprises an RNA polymerase III (pol III) promoter operably linked to the crRNA-encoding nucleotide sequence.

In some embodiments of the above aspect, the pol III promoter comprises a nucleotide sequence having at least 90% sequence identity to the nucleotide sequence set forth as any one of SEQ ID NOs: 1-10. In some embodiments, the pol III promoter comprises a nucleotide sequence having at least 95% sequence identity to the nucleotide sequence set forth as any one of SEQ ID NOs: 1-10. In some embodiments, the pol III promoter comprises a nucleotide sequence having at least 96%, 97%, 98%, or at least 99% sequence identity to the nucleotide sequence set forth as any one of SEQ ID NOs: 1-10. In some embodiments, the pol III promoter comprises a nucleotide sequence having 100% sequence identity to the nucleotide sequence set forth as any one of SEQ ID NOs: 1-10.

In some embodiments of the above aspect, the polynucleotide further comprises an RNA-guided nuclease (RGN)-encoding nucleotide sequence, wherein the RGN is capable of binding to the crRNA and the tracrRNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA comprises a gene silencing RNA.

In still another aspect, the present disclosure provides a vector comprising the expression construct as described hereinabove or the polynucleotide as described hereinabove.

In some embodiments of the above aspect, the vector is a viral vector. In some embodiments, the viral vector is an adeno-associated viral (AAV) vector.

In yet another aspect, the present disclosure provides an expression construct comprising: a) a promoter comprising a nucleotide sequence having at least 90% sequence identity to the nucleotide sequence set forth as any one of SEQ ID NOs: 1-5; and b) a heterologous coding sequence operably linked to the promoter. In some embodiments, the promoter comprises a nucleotide sequence having at least 95% sequence identity to the nucleotide sequence set forth as any one of SEQ ID NOs: 1-5. In some embodiments, the promoter comprises a nucleotide sequence having at least 96%, 97%, 98%, or at least 99% sequence identity to the nucleotide sequence set forth as any one of SEQ ID NOs: 1-5. In some embodiments, the promoter comprises a nucleotide sequence having 100% sequence identity to the nucleotide sequence set forth as any one of SEQ ID NOs: 1-5.

In some embodiments of the above aspect, the promoter comprises a TATA box sequence. In some embodiments, the promoter comprises an octamer (OCT) sequence. In some embodiments, the promoter comprises a SPH sequence. In some embodiments, the promoter comprises a proximal sequence element (PSE).

In some embodiments of the above aspect, the heterologous coding sequence encodes an RNA. In some embodiments, the RNA comprises a guide RNA.

In some embodiments of the above aspect, the heterologous coding sequence encodes a polypeptide.

In another aspect, the present disclosure provides a polynucleotide comprising the expression construct as described hereinabove and an RNA-guided nuclease (RGN)-encoding nucleotide sequence, wherein the RGN is capable of binding the guide RNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA is a crRNA.

In another aspect, the present disclosure provides a polynucleotide comprising the expression construct as described hereinabove and a tracrRNA-encoding nucleotide sequence.

In some embodiments of the above aspect, the polynucleotide further comprises an RNA polymerase III (pol III) promoter operably linked to the tracrRNA-encoding nucleotide sequence.

In some embodiments of the above aspect, the pol III promoter comprises a nucleotide sequence having at least 90% sequence identity to the nucleotide sequence set forth as any one of SEQ ID NOs: 1-10. In some embodiments, the pol III promoter comprises a nucleotide sequence having at least 95% sequence identity to the nucleotide sequence set forth as any one of SEQ ID NOs: 1-10. In some embodiments, the pol III promoter comprises a nucleotide sequence having at least 96%, 97%, 98%, or at least 99% sequence identity to the nucleotide sequence set forth as any one of SEQ ID NOs: 1-10. In some embodiments, the pol III promoter comprises a nucleotide sequence having 100% sequence identity to the nucleotide sequence set forth as any one of SEQ ID NOs: 1-10.

In some embodiments of the above aspect, the polynucleotide further comprises an RNA-guided nuclease (RGN)-encoding nucleotide sequence, wherein the RGN is capable of binding the crRNA and the tracrRNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA is a tracrRNA.

In another aspect, the present disclosure provides a polynucleotide comprising the expression construct as described hereinabove and a crRNA-encoding nucleotide sequence.

In some embodiments of the above aspect, the polynucleotide further comprises an RNA polymerase III (pol III) promoter operably linked to the crRNA-encoding nucleotide sequence.

In some embodiments of the above aspect, the pol III promoter comprises a nucleotide sequence having at least 90% sequence identity to the nucleotide sequence set forth as any one of SEQ ID NOs: 1-10. In some embodiments, the pol III promoter comprises a nucleotide sequence having at least 95% sequence identity to the nucleotide sequence set forth as any one of SEQ ID NOs: 1-10. In some embodiments, the pol III promoter comprises a nucleotide sequence having at least 96%, 97%, 98%, or at least 99% sequence identity to the nucleotide sequence set forth as any one of SEQ ID NOs: 1-10. In some embodiments, the pol III promoter comprises a nucleotide sequence having 100% sequence identity to the nucleotide sequence set forth as any one of SEQ ID NOs: 1-10.

In some embodiments of the above aspect, the polynucleotide further comprises an RNA-guided nuclease (RGN)-encoding nucleotide sequence, wherein the RGN is capable of binding to the crRNA and the tracrRNA.

In some embodiments of the above aspect, the polynucleotide further comprises an RNA polymerase II (pol II) promoter operably linked to the RGN-encoding nucleotide sequence.

In some embodiments of the above aspect, the RGN is fused to a base-editing polypeptide. In some embodiments, the base-editing polypeptide comprises a cytosine deaminase or adenine deaminase.

In some embodiments of the above aspect, the RGN is fused to a transcriptional activator or transcriptional repressor.

In some embodiments of the above aspect, the RGN is fused to a detectable label.

In some embodiments of the above aspect, the RNA comprises a gene silencing RNA.

In yet another aspect, the present disclosure provides a vector comprising the expression construct as described hereinabove or the polynucleotide as described hereinabove.

In some embodiments of the above aspect, the vector is a viral vector. In some embodiments, the viral vector is an adeno-associated viral (AAV) vector.

In still another aspect, the present disclosure provides a cell comprising the promoter as described hereinabove, the expression construct as described hereinabove, the polynucleotide as described hereinabove, or the vector as described hereinabove.

In some embodiments of the above aspect, the cell is a prokaryotic cell.

In some embodiments of the above aspect, the cell is a eukaryotic cell.

In some embodiments of the above aspect, the eukaryotic cell is a mammalian cell. In some embodiments, the mammalian cell is a human cell. In some embodiments, the human cell is an immune cell. In some embodiments, the immune cell is a stem cell. In some embodiments, the stem cell is an induced pluripotent stem cell. In some embodiments, the eukaryotic cell is an insect or avian cell. In some embodiments, the eukaryotic cell is a fungal cell.

In some embodiments of the above aspect, the eukaryotic cell is a plant cell.

In another aspect, the present disclosure provides a plant comprising the promoter as described hereinabove, the expression construct as described hereinabove, the polynucleotide as described hereinabove, the vector as described hereinabove, or the plant cell as described hereinabove.

In another aspect, the present disclosure provides a seed comprising the plant cell as described hereinabove.

In another aspect, the present disclosure provides a pharmaceutical composition comprising a pharmaceutically acceptable carrier and the expression construct as described hereinabove, the polynucleotide as described hereinabove, the vector as described hereinabove, or the cell as described hereinabove.

In some embodiments of the above aspect, the pharmaceutical composition is lipid-based. In some embodiments of the above aspect, the lipid-based pharmaceutical composition comprises liposomes or lipid nanoparticles (LNPs). In some embodiments of the above aspect, the expression construct, the polynucleotide, the vector, or the cell is encapsulated in, and/or non-covalently or covalently attached to the liposomes or the LNPs.

In another aspect, the present disclosure provides a method for preparing an expression construct wherein the method comprises inserting a coding sequence into a nucleic acid molecule comprising a promoter as described hereinabove such that the coding sequence is operably linked to the promoter.

In another aspect, the present disclosure provides a method for preparing an expression construct wherein the method comprises inserting a promoter comprising a nucleotide sequence having at least 90% sequence identity to the nucleotide sequence set forth as any one of SEQ ID NOs: 6-10 into a nucleic acid molecule comprising a coding sequence such that the coding sequence is operably linked to the promoter.

In some embodiments of the above aspect, the promoter comprises a nucleotide sequence having at least 95% sequence identity to the nucleotide sequence set forth as any one of SEQ ID NOs: 6-10. In some embodiments, the promoter comprises a nucleotide sequence having at least 96%, 97%, 98%, or at least 99% sequence identity to the nucleotide sequence set forth as any one of SEQ ID NOs: 6-10. In some embodiments, the promoter comprises a nucleotide sequence having 100% sequence identity to the nucleotide sequence set forth as any one of SEQ ID NOs: 6-10.

In some embodiments of the above aspect, the promoter comprises a TATA box sequence. In some embodiments, the promoter comprises an octamer (OCT) sequence. In some embodiments, the promoter comprises a SPH sequence. In some embodiments, the promoter comprises a proximal sequence element (PSE).

In some embodiments of the above aspect, the coding sequence is heterologous to the promoter.

In some embodiments of the above aspect, the coding sequence encodes an RNA. In some embodiments of the above aspect, the RNA comprises a guide RNA.

In some embodiments of the above aspect, the RNA is a crRNA.

In some embodiments of the above aspect, the RNA is a tracrRNA.

In some embodiments of the above aspect, the RNA comprises a gene silencing RNA.

In some embodiments of the above aspect, the coding sequence encodes a polypeptide.

In still another aspect, the present disclosure provides a method for expressing an RNA, the method comprising contacting in vitro the expression construct as described hereinabove, or the polynucleotide as described hereinabove with RNA polymerase III and ribonucleotide triphosphates.

In still another aspect, the present disclosure provides a method for expressing an RNA in a cell, the method comprising introducing into the cell the expression construct as described hereinabove, or the polynucleotide as described hereinabove.

In yet another aspect, the present disclosure provides a method for expressing a polypeptide in a cell, the method comprising introducing into the cell the expression construct as described hereinabove.

In yet another aspect, the present disclosure provides a method for making an RGN ribonucleoprotein complex in a cell, the method comprising introducing into a cell the polynucleotide as described hereinabove, wherein the RGN and the gRNA form an RGN ribonucleoprotein complex.

In some embodiments of the above aspect, the method further comprises purifying the RGN ribonucleoprotein complex.

In still another aspect, the present disclosure provides a method for making an mRNA in vitro, the method comprising contacting the expression construct as described hereinabove with RNA polymerase II and ribonucleotide triphosphates.

In some embodiments of the above aspect, the method further comprises contacting the expression construct with a cap analog to generate a capped mRNA. In some embodiments, the method further comprises contacting the capped mRNA with a poly(A) polymerase to generate a capped and tailed mRNA. In some embodiments, the method further comprises contacting the capped mRNA or the capped and tailed mRNA in vitro with a cell-free lysate to generate an in vitro translated polypeptide.

In still another aspect, the present disclosure provides a method for binding a target nucleic acid molecule in a cell, wherein said target nucleic acid molecule comprises a target sequence, the method comprising introducing into the cell the polynucleotide as described hereinabove, wherein the guide RNA as described hereinabove or the crRNA and tracrRNA of the polynucleotide as described hereinabove binds to the RGN, thereby directing the RGN to bind to the target sequence within said target nucleic acid molecule.

In still another aspect, the present disclosure provides a method for cleaving and/or modifying a target nucleic acid molecule in a cell, wherein said target nucleic acid molecule comprises a target sequence, the method comprising introducing into the cell the polynucleotide as described hereinabove, wherein the guide RNA as described hereinabove or the crRNA and tracrRNA of the polynucleotide as described hereinabove binds to the RGN, thereby directing the RGN to bind to the target sequence and cleave and/or modify the target nucleic acid molecule.

In yet another aspect, the present disclosure provides a method for modifying the expression of a target gene in a cell, wherein the target gene comprises a target sequence, the method comprising introducing into the cell the polynucleotide as described hereinabove, wherein the guide RNA as described hereinabove or the crRNA and tracrRNA of the polynucleotide as described hereinabove binds to the RGN, thereby directing the RGN to the target sequence and modifying the expression of the target gene.

In still another aspect, the present disclosure provides a method for detecting a target sequence in a cell, the method comprising introducing into the cell the polynucleotide as described hereinabove, wherein the guide RNA as described hereinabove or the crRNA and tracrRNA of the polynucleotide as described hereinabove binds to the RGN, thereby directing the RGN to the target sequence; and detecting the detectable label.

In yet another aspect, the present disclosure provides a method for binding a target nucleic acid molecule in a cell, wherein said target nucleic acid molecule comprises a target sequence, wherein the method comprises introducing into the cell the expression construct as described hereinabove, or the polynucleotide as described hereinabove, wherein the cell comprises an RNA-guided nuclease (RGN); and wherein the guide RNA as described hereinabove, or the crRNA and tracrRNA of the polynucleotide as described hereinabove binds to the RGN, thereby directing the RGN to bind the target sequence within the target nucleic acid molecule.

In another aspect, the present disclosure provides a method for cleaving and/or modifying a target nucleic acid molecule in a cell, wherein the target nucleic acid molecule comprises a target sequence, wherein the method comprises introducing into the cell the expression construct as described hereinabove, or the polynucleotide as described hereinabove, wherein the cell comprises: a) an RNA-guided nuclease (RGN); or b) a fusion protein comprising a nuclease-inactive or nickase RGN fused to a base-editing polypeptide; and wherein the guide RNA as described hereinabove, or the crRNA and tracrRNA of the polynucleotide as described hereinabove binds to the RGN, thereby directing the RGN or the fusion protein to bind to the target sequence and cleave and/or modify the target nucleic acid molecule.

In some embodiments of the above aspect, the method further comprises introducing the RGN or the fusion protein or a nucleic acid molecule encoding the same into the cell prior to or simultaneously with the introduction of the expression construct as described hereinabove, or the polynucleotide as described hereinabove.

In another aspect, the present disclosure provides a method for modifying the expression of a target gene, wherein the target gene comprises a target sequence, wherein the method comprises introducing into the cell the expression construct as described hereinabove, or the polynucleotide as described hereinabove, wherein the cell comprises a fusion protein comprising an RNA-guided nuclease (RGN) fused to a transcriptional activator or transcriptional repressor, and wherein the guide RNA as described hereinabove, or the crRNA and tracrRNA of the polynucleotide as described hereinabove binds to the RGN, thereby directing the fusion protein to the target sequence and modifying the expression of the target gene.

In some embodiments of the above aspect, the method further comprises introducing the fusion protein or a nucleic acid molecule encoding the same into the cell prior to or simultaneously with the introduction of the expression construct as described hereinabove, or the polynucleotide as described hereinabove.

In another aspect, the present disclosure provides a method for detecting a target sequence in a cell, wherein the method comprises introducing into the cell the expression construct as described hereinabove, or the polynucleotide as described hereinabove, wherein the cell comprises a fusion protein comprising an RNA-guided nuclease (RGN) fused to a detectable label, and wherein the guide RNA as described hereinabove, or the crRNA and tracrRNA of the polynucleotide as described hereinabove binds to the RGN, thereby directing the fusion protein to the target sequence; and detecting the detectable label.

In some embodiments of the above aspect, the method further comprises introducing the fusion protein or a nucleic acid molecule encoding the same into the cell prior to or simultaneously with the introduction of the expression construct as described hereinabove, or the polynucleotide as described hereinabove.

In still another aspect, the present disclosure provides a method for binding a target nucleic acid molecule in a cell, wherein said target nucleic acid molecule comprises a target sequence, wherein the method comprises: a) introducing into the cell the expression construct as described hereinabove, or the polynucleotide as described hereinabove; and b) introducing into the cell: i) an RNA-guided nuclease (RGN); or ii) a nucleic acid molecule encoding an RGN; wherein the step a) occurs before, during, or after step b); and wherein the guide RNA as described hereinabove, or the crRNA and tracrRNA of the polynucleotide as described hereinabove binds to the RGN, thereby directing the RGN to bind the target sequence within the target nucleic acid molecule.

In still another aspect, the present disclosure provides a method for cleaving and/or modifying a target nucleic acid molecule in a cell, wherein the target nucleic acid molecule comprises a target sequence, wherein the method comprises: a) introducing into the cell the expression construct as described hereinabove, or the polynucleotide as described hereinabove; and b) introducing into the cell: i) an RNA-guided nuclease (RGN); ii) a fusion protein comprising a nuclease-inactive or nickase RGN fused to a base-editing polypeptide; iii) a nucleic acid molecule encoding an RGN; or iv) a nucleic acid molecule encoding a fusion protein comprising a nuclease-inactive or nickase RGN fused to a base-editing polypeptide; wherein the step a) occurs before, during, or after step b); and wherein the guide RNA as described hereinabove, or the crRNA and tracrRNA of the polynucleotide as described hereinabove binds to the RGN, thereby directing the RGN or the fusion protein to bind to the target sequence and cleave and/or modify the target nucleic acid molecule.

In some embodiments of the above aspect, the step b) iii) comprises introducing a polynucleotide comprising the nucleic acid molecule encoding the RGN or the step b) iv) comprises introducing a polynucleotide comprising the nucleic acid molecule encoding the fusion protein, and wherein the polynucleotide further comprises an RNA polymerase II (pol II) promoter operably linked to the nucleic acid molecule encoding the RGN or the nucleic acid molecule encoding the fusion protein.

In yet another aspect, the present disclosure provides a method for modifying the expression of a target gene, wherein the target gene comprises a target sequence, wherein the method comprises: a) introducing into the cell the expression construct as described hereinabove, or the polynucleotide as described hereinabove; and b) introducing into the cell: i) a fusion protein comprising a nuclease-inactive RNA-guided nuclease (RGN) fused to a transcriptional activator or transcriptional repressor; or ii) a nucleic acid molecule encoding a fusion protein comprising a nuclease-inactive RGN fused to a transcriptional activator or transcriptional repressor; wherein the step a) occurs before, during, or after step b); and wherein the guide RNA as described hereinabove, or the crRNA and tracrRNA of the polynucleotide as described hereinabove binds to the RGN, thereby directing the fusion protein to the target sequence and modifying the expression of the target gene.

In some embodiments of the above aspect, the step b) ii) comprises introducing a polynucleotide comprising the nucleic acid molecule encoding the fusion protein, and wherein the polynucleotide further comprises an RNA polymerase II (pol II) promoter operably linked to the nucleic acid molecule encoding the fusion protein.

In yet another aspect, the present disclosure provides a method for detecting a target sequence in a cell, wherein the method comprises: a) introducing into the cell the expression construct as described hereinabove, or the polynucleotide as described hereinabove; b) introducing into the cell: i) a fusion protein comprising nuclease-inactive RNA-guided nuclease (RGN) fused to a detectable label; or ii) a nucleic acid molecule encoding a fusion protein comprising a nuclease-inactive RGN fused to a detection label; and c) detecting the detectable label; wherein step a) occurs before, during, or after step b); and wherein the guide RNA as described hereinabove, or the crRNA and tracrRNA of the polynucleotide as described hereinabove binds to the RGN and directs the fusion protein to the target sequence.

In some embodiments of the above aspect, the step b) ii) comprises introducing into the cell a polynucleotide comprising the nucleic acid molecule encoding the fusion protein, and wherein the polynucleotide further comprises an RNA polymerase II (pol II) promoter operably linked to the nucleic acid molecule encoding the fusion protein.

In another aspect, the present disclosure provides a method for reducing the expression of a target gene in a cell, the method comprising introducing the expression construct as described hereinabove, wherein the gene silencing RNA reduces the expression of the target gene.

In another aspect, the present disclosure provides a method for treating a disease, the method comprising administering to a subject in need thereof a pharmaceutical composition comprising a pharmaceutically acceptable carrier and a polynucleotide as described hereinabove.

In some embodiments of the above aspect, the disease is associated with a causal mutation and the pharmaceutical composition corrects the causal mutation.

In another aspect, the present disclosure provides use of the polynucleotide as described hereinabove for the treatment of a disease in a subject.

In some embodiments of the above aspect, the disease is associated with a causal mutation and the treatment comprises correcting the causal mutation.

In another aspect, the present disclosure provides use of the polynucleotide as described hereinabove for the manufacture of a medicament useful for treating a disease.

In some embodiments of the above aspect, the disease is associated with a causal mutation and the medicament corrects the causal mutation.

In still another aspect, the present disclosure provides a method for treating a disease, the method comprising administering to a subject in need thereof a pharmaceutical composition comprising a pharmaceutically acceptable carrier and a polynucleotide as described hereinabove, wherein the disease is associated with the expression of a target gene, wherein the target gene comprises a target sequence, and wherein the guide RNA of the polynucleotide as described hereinabove or the crRNA and tracrRNA of the polynucleotide as described hereinabove binds to the RGN, thereby directing the RGN to the target sequence and modifying the expression of the target gene.

In still another aspect, the present disclosure provides use of the polynucleotide as described hereinabove for the treatment of a disease associated with the expression of a target gene in a subject.

In yet another aspect, the present disclosure provides use of the polynucleotide as described hereinabove for the manufacture of a medicament useful for treating a disease associated with the expression of a target gene.

In yet another aspect, the present disclosure provides a method for treating a disease, the method comprising administering to a subject in need thereof a pharmaceutical composition comprising a pharmaceutically acceptable carrier and an expression construct as described hereinabove, wherein the disease is associated with the overexpression of a target gene, and wherein the gene silencing RNA reduces the expression of the target gene.

In still another aspect, the present disclosure provides use of the expression construct as described hereinabove for the treatment of a disease associated with the overexpression of a target gene in a subject.

In still another aspect, the present disclosure provides use of the expression construct as described hereinabove for the manufacture of a medicament useful for treating a disease associated with the overexpression of a target gene.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

Nucleic acids comprising a promoter and expression cassettes wherein the promoters are operably linked to a coding sequence are provided. A promoter is a non-coding DNA sequence sufficient to direct transcription of an operably linked nucleic acid molecule, which is usually downstream (3′) of the promoter. The term “promoter” encompasses a minimal promoter, the minimal sequence needed for RNA polymerase binding and initiating transcription of an operably linked coding sequence, as well as transcription control elements (e.g., enhancers) that render promoter-dependent gene expression controllable in a cell type-specific, tissue-specific, or temporal-specific manner, or that are inducible by signals or agents.

In eukaryotes, the nucleotide sequence of the promoter determines the type of RNA polymerase (i.e., RNA polymerase I, RNA polymerase II, or RNA polymerase III) that binds and initiates transcription at a specific transcription initiation site. In general, RNA polymerase I transcribes ribosomal RNA (except for ribosomal 5S rRNA), RNA polymerase II transcribes DNA into precursors of messenger RNA (mRNA) that can then be translated into polypeptides, most small nuclear (snRNA), and microRNA, and RNA polymerase III transcribes DNA into ribosomal 5S rRNA, tRNA, and other small RNAs such as the U6 snRNA.

The presently disclosed promoters can function as pol III promoters. RNA polymerase III promoters, also referred to herein as pol III promoters, are promoters that are capable of being bound by RNA polymerase III (pol III), which then initiates transcription of an operably linked RNA-coding sequence. In some instances, pol III promoters can also be bound by RNA polymerase II (pol II), which can initiate transcription of mRNA (see, for example, Gao et al. (2018) Molecular Therapy: Nucleic Acids 12:135-145). Thus, in some embodiments, the presently disclosed promoters can be bound by pol II and transcription initiated therefrom. The presently disclosed promoters can be used to initiate transcription of an RNA-encoding sequence through the recruitment of pol III or of a polypeptide-encoding sequence through the recruitment of pol II.

As used herein, a “coding sequence” refers to a nucleotide sequence that encodes either an RNA (e.g., rRNA, tRNA, snRNA, microRNA, guide RNA) or a messenger RNA (mRNA) precursor that can then be translated into a polypeptide comprising an amino acid sequence. Coding sequences that encode an RNA that cannot be translated into a polypeptide are referred to herein as RNA-encoding sequences. In general, when operably linked to the presently disclosed promoters, RNA-encoding sequences are transcribed by pol III. Coding sequences that encode a mRNA precursor that can then be translated into a polypeptide are referred to herein as polypeptide-encoding sequences. Such polypeptide-encoding sequences are transcribed by pol II.

### II. Promoter Sequences

Provided herein are promoter sequences having the sequence set forth in any one of SEQ ID NOs: 1-10 or an active variant or fragment thereof. The presently disclosed promoters are RNA pol III promoters and in some embodiments, can function as RNA pol II promoters by recruiting pol II.

There are three types of pol III promoters: type 1, type 2, and type 3. Type 1 pol III promoters include 5S rRNA promoters and initiation involves TFIIIA binding to the C Block intragenic 5S rRNA control sequence, serving as a platform to position TFIIIC, and subsequent assembly of TFIIIB. Type 2 pol III promoters include tRNA promoters and transcription initiation involves TFIIIC binding to A and B Block intragenic regions, serving as a platform to position TFIIIB, which in turn, assembles Pol III at the transcription start site. Type 3 pol III promoters solely utilize upstream regulatory elements and do not utilize intragenic elements. Type 3 pol III promoters generally comprise a proximal sequence element (PSE), a TATA box, and an upstream distal sequence element (DSE). The DSE comprises at least one of an octamer (OCT) sequence and a SphI postoctamer homology (SPH) sequence. The transcription factor Oct-1 binds the OCT sequence in the DSE and a SBF (SPH-binding factor) (also referred to as STAF (selenocysteine tRNA gene transcription activating factor)/SBF or zinc finger protein 143 (ZNF143)) transcription factor binds to the SPH sequence in the DSE. Assembly of the snRNA activating protein complex (SNAPc) to the PSE is stimulated by Octi and/or SBH binding to the DSE. SNAPc acts to assemble TFIIIB, which consists of a TATA-binding protein (TBP), a TFIIB-related factor (BRF2), and B-double-prime (BDP1), at the TATA box. TFIIIB, in turn, assembles Pol III at the start site of transcription. Type 3 pol III promoters, such as 7SK, U6, and H1, can be used for the expression of small RNA, including short hairpin RNA (shRNA) and guide RNA (gRNA).

In some embodiments, the presently disclosed promoters or active variants or fragments thereof do not comprise intragenic elements for the regulation of transcription of an operably linked sequence and are thus considered type 3 pol III promoters.

In some embodiments, active variants or fragments of the presently disclosed promoters comprise a TATA box sequence that is recognized by a TATA-binding protein. The consensus sequence of a TATA box is generally TATAWAW (SEQ ID NO: 54), wherein W is A or T. In some embodiments, active variants or fragments of the presently disclosed promoters comprise a TATA box having the sequence of TATAA (SEQ ID NO: 55).

In some embodiments, active variants or fragments of the presently disclosed promoters comprise an OCT sequence that is recognized and bound by an Oct-1 transcription factor. Oct-1 transcription factors belong to the POU transcription factor family and comprise a 160-amino acid POU domain necessary for DNA binding to an octameric sequence. A non-limiting example of an Oct-1 transcription factor is the human Oct-1 set forth as NCBI Acc. No. NP_001352777. In some embodiments, the OCT sequence comprises the consensus sequence of ATTTGCAT (SEQ ID NO: 56).

In some embodiments, active variants or fragments of the presently disclosed promoters comprise a SPH sequence that is recognized and bound by a SPH-binding factor (SBF) transcription factor, which is also referred to as the zinc finger 143 (ZNF143) protein and comprises a zinc finger binding domain. A non-limiting example of a SBF protein is the human SBF/ZNF143 protein set forth as NCBI Acc. No. NP_003433.3. In some embodiments, the SPH sequence comprises the consensus sequence of CTCCGCCCCGCTTCCGG (SEQ ID NO: 57).

In some embodiments, active variants or fragments of the presently disclosed promoters comprise a PSE that is recognized and bound by the SNAPc complex. The SNAPc complex is comprised of five subunits, SNAP190, SNAP50/PTFβ, SNAP45/PTFδ, SNAP43/PTFγ, and SNAP19 (see, e.g., Henry et al. 1998 Cold Spring Harbor Symp. Quant. Biol. Vol. 63, pp. 111-120, which is herein incorporated by reference in its entirety). The PSE is often approximately 55 bp upstream from the transcription start site, but can range from about −66 bp to about −47 bp (see, e.g., Myslinski et al. 2001 Nucleic Acids Res 29(12):2502-2509, which is herein incorporated by reference). In some embodiments, the PSE is between −66 bp and −47 bp (i.e., between 66 bp and 47 bp upstream from the transcription start site).

In some instances, pol III promoters can also be bound by pol II, which can initiate transcription of mRNA (Gao et al. (2018) Molecular Therapy: Nucleic Acids 12:135-145). Thus, in some embodiments, the presently disclosed promoters can function as pol II promoters by recruiting pol II and allowing transcription to be initiated therefrom.

In some embodiments, the promoter is truncated through the removal of sequence, such as the sequence that is looped around a positional nucleosome. The promoters set forth as SEQ ID NOs: 6-10 are truncated versions of SEQ ID NOs: 1-5, respectively, in which the putative nucleosomal sequence has been removed, as well as nucleotides from the 5′ end of the promoter.

### III. Vectors and Expression Constructs

Provided herein are polynucleotides and vectors comprising a presently disclosed promoter (i.e., SEQ ID NOs: 1-10 or an active variant or fragment of any thereof) and expression constructs that comprise a presently disclosed promoter operably linked to a coding sequence.

The use of the term “polynucleotide” or “nucleic acid molecule” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

The polynucleotide comprising a presently disclosed promoter (i.e., SEQ ID NOs: 1-10 or an active variant or fragment of any thereof) can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

The presently disclosed promoters (i.e., SEQ ID NOs: 1-10 or an active variant or fragment of any thereof) can be provided in expression constructs (also referred to herein as “expression cassettes”) for in vitro expression or expression in a cell, organelle, embryo, or organism of interest. In various embodiments, the cassette includes 5′, and in some embodiments, 3′ regulatory sequences operably linked to a polynucleotide encoding an RNA or a polypeptide that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding sequence of interest (e.g., region coding for an RNA or a polypeptide) is a functional link that allows for expression of the coding sequence of interest. Operably linked elements may be contiguous or non-contiguous. When used to refer to the joining of two protein coding regions, by operably linked is intended that the coding regions are in the same reading frame. Alternatively, the additional gene(s) or element(s) can be provided on multiple expression cassettes. For example, a nucleotide sequence encoding an RNA-guided nuclease (RGN) can be present on one expression cassette, whereas a nucleotide sequence encoding an RNA (e.g., guide RNA) can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter such as those disclosed herein), a coding sequence, and a transcriptional (and in some embodiments, translational) termination region (i.e., termination region) functional in the organism of interest. In some embodiments, the transcriptional termination region comprises a poly T stretch. In some embodiments, the poly T stretch comprises at least 4 thymines.

The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions for polypeptide-encoding sequences are available from the Ti-plasmid of A. tumefaciens, such as the octopine synthase and nopaline synthase termination regions. See also Guerineau et al. (1991)Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In some embodiments, a coding sequence for a polypeptide (e.g., an RGN) also can be linked to a polyadenylation signal (e.g., SV40 polyA signal and other signals functional in plants) and/or at least one transcriptional termination sequence. Additionally, the coding sequence for a polypeptide (e.g., an RGN) also can be linked to sequence(s) encoding at least one nuclear localization signal, at least one cell-penetrating domain, and/or at least one signal peptide capable of trafficking proteins to particular subcellular locations, as described elsewhere herein.

In some embodiments, a polynucleotide may comprise two expression constructs, one of which expresses a crRNA and another that expresses a tracrRNA. At least one of the crRNA- and tracrRNA-encoding sequences is operably linked to a presently disclosed promoter (i.e., SEQ ID NOs: 1-10 or an active variant or fragment of any thereof). In some embodiments, one of the crRNA- and tracrRNA-encoding sequences is operably linked to a presently disclosed promoter and the other coding sequence is operably linked to another pol III promoter. Examples of suitable Pol III promoters include, but are not limited to, mammalian U6, U3, H1, and 7SL RNA promoters and rice U6 and U3 promoters.

In some embodiments, the expression cassette or vector comprising a sequence encoding a crRNA and/or a tracrRNA, or the crRNA and tracrRNA combined to create a guide RNA, can further comprise a sequence encoding an RGN polypeptide that is capable of binding to the crRNA and/or tracrRNA. In some embodiments, the RGN-encoding sequence is operably linked to a pol II promoter. Given that the presently disclosed promoters may recruit pol II, in some embodiments, the RGN-encoding sequence is operably linked to a presently disclosed promoter. In other embodiments, the RGN-encoding sequence is operably linked to a pol II promoter other than the presently disclosed promoters (i.e., SEQ ID NOs: 1-10 or an active variant or fragment of any thereof).

### IV. Coding Sequences

The presently disclosed promoters (i.e., SEQ ID NOs: 1-10 or an active variant or fragment of any thereof) can be used to express an RNA via the recruitment of pol III or a polypeptide by pol II. Thus, in some embodiments, a presently disclosed promoter is operably linked to a RNA coding sequence. In other embodiments, a presently disclosed promoter is operably linked to a polypeptide-encoding sequence.

### A. RNA Coding Sequences

In some embodiments, a presently disclosed promoter (i.e., any one of SEQ ID NOs: 1-10 or an active variant or fragment of any thereof) is operably linked to a coding sequence that encodes an RNA. As used herein, “a coding sequence that encodes an RNA” does not encompass a sequence that can be transcribed into an mRNA or a pre-mRNA that can then be translated into a polypeptide. These coding sequences are referred to herein as coding sequences that encode a polypeptide or “polypeptide-encoding sequences”.

The RNA molecule that can be encoded by an RNA coding sequence include any type of RNA (excluding an mRNA or pre-mRNA), including but not limited to an snRNA, a tRNA, an RNA that can be used in an RNA-guided nuclease system (e.g., a crRNA, a tracrRNA, and a guide RNA), and an RNA that can be used in gene silencing applications (e.g., shRNA, siRNA, antisense RNA).

i. crRNA, tracrRNA, Guide RNA

The presently disclosed promoters (i.e., SEQ ID NOs: 1-10 or an active variant or fragment of any thereof) can be used to express RNAs involved in RNA-guided nuclease systems that when bound to an RGN, can be used to bind to and/or modify a nucleotide sequence. A presently disclosed promoter can be operably linked to a crRNA-encoding sequence, a tracrRNA-encoding sequence, or a guide RNA-encoding sequence.

The term “guide RNA” refers to a nucleotide sequence having sufficient complementarity with a target nucleotide sequence to hybridize with the target sequence and direct sequence-specific binding of an associated RNA-guided nuclease to the target nucleotide sequence. More specifically, when the target nucleotide sequence is double-stranded as is the case with DNA, the target nucleotide sequence is comprised of a target strand (which comprises the PAM sequence) and the non-target strand. In these embodiments, the guide RNA has sufficient complementarity with the non-target strand of a double-stranded target sequence (e.g., target DNA) such that the guide RNA hybridizes with the non-target strand and directs sequence-specific binding of an associated RNA-guided nuclease (RGN) to the target sequence (e.g., target DNA sequence). Therefore, in some embodiments, a guide RNA includes a spacer that is identical to the sequence of the target strand except that uracil (U) replaces thymidine (T) in the guide RNA.

An RGN's respective guide RNA is one or more RNA molecules (generally, one or two), that can bind to the RGN and guide the RGN to bind to a particular target nucleotide sequence, and in those embodiments wherein the RGN has nickase or nuclease activity, also cleave the target strand and/or the non-target strand. In general, a guide RNA comprises a CRISPR RNA (crRNA) and a trans-activating CRISPR RNA (tracrRNA), although some RGNs do not require a tracrRNA. Native guide RNAs that comprise both a crRNA and a tracrRNA generally comprise two separate RNA molecules that hybridize to each other through the repeat sequence of the crRNA and the anti-repeat sequence of the tracrRNA.

In some embodiments, a presently disclosed promoter is operably linked to a coding sequence for a CRISPR RNA. A CRISPR RNA (crRNA) comprises a spacer and a CRISPR repeat. The “spacer” has a nucleotide sequence that directly hybridizes with the non-target strand of a target nucleotide sequence of interest. The spacer is engineered to have full or partial complementarity with the non-target strand of a target sequence of interest. In some embodiments, the spacer can comprise from about 8 nucleotides to about 30 nucleotides, or more. For example, the spacer can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer is 8, 9, 10, 11, 12, 13, 14, 15, 16, 17, 18, 19, 20, 21, 22, 23, 24, 25, 26, 27, 28, 29, 30, or more nucleotides in length. In some embodiments, the spacer is about 10 to about 26 nucleotides in length, or about 12 to about 30 nucleotides in length. In some embodiments, the spacer is about 30 nucleotides in length. In some embodiments, the spacer is 10 to 26 nucleotides in length, or 12 to 30 nucleotides in length. In some embodiments, the spacer is 30 nucleotides in length. In some embodiments, the degree of complementarity between a spacer and the non-target strand of a target sequence, when optimally aligned using a suitable alignment algorithm, is between 50% and 99% or more, including but not limited to about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a spacer and the non-target strand of a target sequence, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more. In some embodiments, the spacer can be identical in sequence to the target strand of a target sequence. In some of those embodiments wherein the target sequence is a target DNA sequence, the spacer can be identical in sequence to the target strand of the target DNA sequence, with the exception of the thymidines (Ts) in the target strand being replaced by uracils (Us) in the spacer. In some embodiments, the spacer is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

Along with a spacer, crRNAs further comprise a CRISPR RNA repeat. The CRISPR RNA repeat comprises a nucleotide sequence that forms a structure, either on its own or in concert with a hybridized tracrRNA, that is recognized by the RGN molecule. In some embodiments, the CRISPR RNA repeat can comprise from about 8 nucleotides to about 30 nucleotides, or more. For example, the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR RNA repeat is 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat and its corresponding tracrRNA, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the crRNA that is expressed using the presently disclosed promoters (i.e., SEQ ID NOs: 1-10 or an active variant or fragment of any thereof) is not naturally-occurring. In some of these embodiments, the specific CRISPR repeat is not linked to the engineered spacer in nature and the CRISPR repeat is considered heterologous to the spacer. In some embodiments, the spacer is an engineered sequence that is not naturally occurring.

In some embodiments, a presently disclosed promoter (i.e., any one of SEQ ID NOs: 1-10 or an active variant or fragment of any thereof) is operably linked to a coding sequence for a tracrRNA. A trans-activating CRISPR RNA or tracrRNA molecule comprises a nucleotide sequence comprising a region that has sufficient complementarity to hybridize to a CRISPR repeat of a crRNA, which is referred to herein as the anti-repeat. In some embodiments, the tracrRNA molecule further comprises a region with secondary structure (e.g., stem-loop) or forms secondary structure upon hybridizing with its corresponding crRNA. In some embodiments, the region of the tracrRNA that is fully or partially complementary to a CRISPR repeat is at the 5′ end of the molecule and the 3′ end of the tracrRNA comprises secondary structure. This region of secondary structure generally comprises several hairpin structures, including the nexus hairpin, which is found adjacent to the anti-repeat. The nexus forms the core of the interactions between the guide RNA and the RGN, and is at the intersection between the guide RNA, the RGN, and the target DNA. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In some embodiments, the anti-repeat region of the tracrRNA that is fully or partially complementary to the CRISPR repeat comprises from about 8 nucleotides to about 30 nucleotides, or more. For example, the region of base pairing between the tracrRNA anti-repeat and the CRISPR repeat can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the anti-repeat region of the tracrRNA that is fully or partially complementary to the CRISPR repeat sequence comprises from 8 nucleotides to 30 nucleotides, or more. In some embodiments, the region of base pairing between the tracrRNA anti-repeat and the CRISPR repeat is 6, 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the degree of complementarity between a CRISPR repeat and its corresponding tracrRNA anti-repeat, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In some embodiments, the degree of complementarity between a CRISPR repeat and its corresponding tracrRNA anti-repeat, when optimally aligned using a suitable alignment algorithm, is 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more.

In some embodiments, the entire tracrRNA can comprise from about 60 nucleotides to more than about 210 nucleotides. For example, the tracrRNA can be about 50, about 55, about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210, or more nucleotides in length. In some embodiments, the entire tracrRNA comprises from 60 nucleotides to more than 140 nucleotides. In some embodiments, the tracrRNA is 50, 55, 60, 65, 70, 75, 80, 85, 90, 95, 100, 105, 110, 115, 120, 125, 130, 135, 140, 150, 160, 170, 180, 190, 200, 210, or more nucleotides in length. In some embodiments, the tracrRNA is about 80 to about 90 nucleotides in length, including about 80, about 81, about 82, about 83, about 84, about 85, about 86, about 87, about 88, about 89, and about 90 nucleotides in length. In some embodiments, the tracrRNA is 80 to 90 nucleotides in length, including 80, 81, 82, 83, 84, 85, 86, 87, 88, 89, and 90 nucleotides in length.

Two polynucleotide sequences can be considered to be substantially complementary when the two sequences hybridize to each other under stringent conditions. Likewise, an RGN is considered to bind to a particular target sequence within a sequence-specific manner if the guide RNA bound to the RGN binds to the target sequence under stringent conditions. By “stringent conditions” or “stringent hybridization conditions” is intended conditions under which the two polynucleotide sequences will hybridize to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3, and the temperature is at least about 30° C. for short sequences (e.g., 10 to 50 nucleotides) and at least about 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The term “sequence specific” can also refer to the binding of a RGN polypeptide to a target sequence at a greater frequency than binding to a randomized background sequence.

The presently disclosed promoters (i.e., SEQ ID NOs: 1-10 or an active variant or fragment of any thereof) can be used to express a guide RNA. The guide RNA can be a crRNA only (some RGNs do not require a tracrRNA and the crRNA can serve as a guide RNA), a single guide RNA, or a dual guide RNA. A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA (i.e., the antirepeat), which may be fully or partially complementary to the CRISPR repeat of the crRNA. In some of those embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In some embodiments, the linker nucleotide sequence of a single guide RNA is at least 4 nucleotides in length. In some embodiments, the linker nucleotide sequence is the nucleotide sequence set forth as SEQ ID NO: 249.

The single guide RNA or dual-guide RNA can be synthesized chemically or via in vitro transcription. Assays for determining sequence-specific binding between an RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA:RGN complex is captured via the detectable label (e.g., with streptavidin beads). A control guide RNA with an unrelated sequence or structure to the guide RNA can be used as a negative control for non-specific binding of the RGN to RNA.

Non-limiting examples of crRNAs and/or tracrRNAs that can be expressed with the presently disclosed promoters are provided in Table 1, along with their corresponding RNA-guided nucleases.

In some embodiments, the guide RNA can be introduced into a target cell, organelle, or embryo as an RNA molecule. In some embodiments, the guide RNA can be transcribed in vitro or chemically synthesized. In some of these embodiments, the nucleotide sequence encoding the guide RNA is operably linked to a presently disclosed promoter (i.e., SEQ ID NOs: 1-10 or an active variant or fragment of any thereof). In some embodiments, the promoter is heterologous to the guide RNA-encoding nucleotide sequence.

In some embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence of interest. The target sequence can be bound (and in some embodiments, cleaved) by an RNA-guided nuclease in vitro or in a cell. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided nuclease in vitro or in a cell. In those embodiments wherein the target sequence is a chromosomal sequence, the chromosomal sequence can be a nuclear, plastid, or mitochondrial chromosomal sequence. In some of the presently disclosed compositions and methods, the target sequence is within a target nucleic acid molecule that is double-stranded. In some embodiments, the target nucleotide sequence is unique in the target genome.

In some embodiments, the target nucleotide sequence is adjacent to a protospacer adjacent motif (PAM) and the target strand of the target sequence is the strand that comprises the PAM. While a protospacer adjacent motif can be within about 1 to about 10 nucleotides from the target sequence, including about 1, about 2, about 3, about 4, about 5, about 6, about 7, about 8, about 9, or about 10 nucleotides from the target sequence, generally, the PAM sequence is immediately adjacent to the target sequence and is immediately adjacent unless specified otherwise. In some embodiments, a PAM is within 1 to 10 nucleotides from the target sequence, including 1, 2, 3, 4, 5, 6, 7, 8, 9, or 10 nucleotides from the target sequence. The PAM can be 5′ or 3′ of the target sequence on its target strand. Generally, the PAM is a consensus sequence of about 3-4 nucleotides, but in some embodiments it can be 2, 3, 4, 5, 6, 7, 8, 9, or more nucleotides in length.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell, organelle, or embryo.

Upon recognizing its corresponding PAM sequence, the RGN can cleave one or both strands of a target DNA sequence at a specific cleavage site. As used herein, a cleavage site is made up of the two particular nucleotides within a target sequence between which the strand of a target DNA sequence is cleaved by an RGN. The cleavage site can comprise the 1^{st} and 2^{nd}, 2^{nd} and 3^{rd}, 3^{rd} and 4^{th}, 4^{th} and 5^{th}, 5^{th} and 6^{th}, 7^{th} and 8^{th}, or 8^{th} and 9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. As RGNs can cleave a target DNA sequence resulting in staggered ends, in some embodiments, the cleavage site is defined based on the distance of the two nucleotides from the PAM on the target strand of the target DNA sequence and for the non-target strand, the distance of the two nucleotides from the complement of the PAM.

ii. Gene Silencing RNAs

In addition to guide RNAs, crRNAs, and tracrRNAs, the presently disclosed promoters (i.e., SEQ ID NOs: 1-10 or an active variant or fragment of any thereof) can be used to express other types of RNAs, including those non-polypeptide coding RNAs that are involved in RNA interference and/or gene silencing. Thus, in some embodiments a presently disclosed promoter is operably linked to a gene silencing RNA. As used herein, a “gene silencing RNA” refers to an RNA molecule that serves to inhibit or reduce the expression of a polypeptide-encoding or RNA-encoding gene (e.g., microRNA) through post-transcriptional mechanisms. Non-limiting examples of gene silencing RNAs include a shRNA, siRNA, antisense RNA, or the like.

As used herein, the term “shRNA” or “short hairpin RNA” refers to an artificial RNA molecule comprising a hairpin that can be used to silence gene expression via RNA interference. A shRNA is generally less than 500 nucleotides in length and in some embodiments is less than 400 nucleotides, less than 300 nucleotides, less than 200 nucleotides, or less than 100 nucleotides. Generally, a stretch of at least 15 nucleotides, at least 20 nucleotides, at least 30 nucleotides, at least 50 nucleotides, or at least 100 nucleotides is base paired with a complementary sequence located on the same RNA molecule, wherein said sequence and complementary sequence are separated by an unpaired region which forms a single-stranded loops above the stem structure created by the two regions of base complementarity. In some embodiments, the loop region is 4 to 15 nucleotides in length, including 4, 5, 6, 7, 8, 9, 10, 11, 12, 13, 14, and 15 nucleotides in length. In some embodiments, the stem structure comprising the two regions of base complementarity is 15 to 500 bp long, or 15 to 300 bp long, or 15 to 100 bp long, including but not limited to 15, 16, 17, 18, 19, 20, 21, 22, 23, 24, 25, 30, 35, 40, 45, 50, 55, 60, 65, 70, 75, 80, 85, 90, 95, and 100. The stem region comprising the two regions of base complementarity comprise a homologous sequence and complementary sequence to a target sequence to be inhibited.

As used herein, the term “siRNA” or “small interfering RNA” or “short interfering RNA” refers to a double-stranded non-coding RNA molecule that silences genes through the RNA interference pathway. Typically, siRNAs are 20-27 base pairs in length, similar to miRNA. In some embodiments, siRNA comprise two overhanding nucleotides on each end. The presently disclosed promoters can be used to express siRNA through the individual expression of each strand of an siRNA molecule. Thus, a first presently disclosed promoter can be operably linked to a sequence encoding one strand of an siRNA molecule and a second presently disclosed promoter (which may be identical or different from the first promoter) is operably linked to a sequence encoding the complementary strand of an siRNA molecule.

As used herein, the term “antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. According to the present invention, an antisense RNA is transcribed from a coding sequence through the operable linkage of the coding sequence with a presently disclosed promoter. In some embodiments, the antisense RNA is transcribed through the recruitment of pol III. In some embodiments, the antisense RNA is transcribed through the recruitment of pol II.

### B. Polypeptide-Encoding Sequences

Given that the presently disclosed promoters can, in some embodiments, function as a pol II promoter by recruiting RNA polymerase II, in some embodiments, the presently disclosed promoters (i.e., SEQ ID NOs: 1-10 or an active variant or fragment of any thereof) are used to express a polypeptide-encoding sequence and generate a pre-mRNA molecule that can then be processed to add a poly(A) tail and spliced if comprising introns, and eventually translated into a polypeptide. The polypeptide-encoding sequence may comprise introns or splice sites or may be a cDNA lacking splice sites. An example of a canonical splice site is AGGT.

The polypeptide-encoding sequence can be codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gown (1990) Plant Physiol. 92:1-11 for a discussion of plant-preferred codon usage). Methods are available in the art for synthesizing plant-preferred genes or mammalian (for example human) codon-optimized coding sequences. See, for example, U.S. Pat. Nos. 5,380,831, and 5,436,391, and Murray et al. (1989) Nucleic Acids Res. 17:477-498, herein incorporated by reference.

In some embodiments, the polypeptide-encoding sequence that is operably linked to a presently disclosed promoter is a coding sequence for an RNA-guided nuclease.

### V. RNA-Guided Nucleases

In those embodiments wherein the vector or polynucleotide comprises a presently disclosed promoter (i.e., SEQ ID NOs: 1-10 or an active variant or fragment of any thereof) operably linked to a coding sequence that encodes a crRNA, a tracrRNA, or a guideRNA, the vector or polynucleotide can further comprise a coding sequence for an RNA-guided nuclease, such as one capable of binding to the guide RNA, crRNA, or tracrRNA. In some of these embodiments, the coding sequence for the RGN is operably linked to a pol II promoter. In some of these embodiments, the pol II promoter is a presently disclosed promoter or active variant thereof capable of recruiting pol II. In other embodiments, the pol II promoter used to express the RGN is another pol II promoter known in the art. The pol II promoter can be selected based on the desired outcome, such as a constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the AdhI promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axig1 promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991)Mol. Gen. Genet. 227:229-237, and U.S. Pat. Nos. 5,814,618 and 5,789,156), herein incorporated by reference.

Tissue-specific or tissue-preferred promoters can be utilized to target expression of an expression construct within a particular tissue. In some embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the coding sequence for an RGN is operably linked to a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

The coding sequence for an RGN can be operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein or a ribonucleoprotein complex can be purified for use in the methods of genome modification described herein.

RNA-guided nucleases (RGNs) allow for the targeted manipulation of specific site(s) within a genome and are useful in the context of gene targeting for therapeutic and research applications. In a variety of organisms, including mammals, RNA-guided nucleases have been used for genome engineering by stimulating non-homologous end joining and homologous recombination, for example. The compositions and methods described herein are useful for creating single- or double-stranded breaks in polynucleotides, modifying polynucleotides, detecting a particular site within a polynucleotide, or modifying the expression of a particular gene.

In some embodiments, the RNA-guided nucleases used in the presently disclosed compositions and methods can alter gene expression by modifying a target gene comprising a target sequence. In specific embodiments, RNA-guided nucleases are directed to the target sequence by a guide RNA (gRNA) as part of a Clustered Regularly Interspaced Short Palindromic Repeats (CRISPR) RNA-guided nuclease system. RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence. After the target sequence has been cleaved, the break can be repaired such that the DNA sequence of the target sequence is modified during the repair process. Thus, provided herein are methods for using the presently disclosed compositions comprising RNA-guided nucleases or nucleic acids encoding the same to modify a target sequence in the DNA of host cells. For example, RNA-guided nucleases can be used to modify a target sequence at a genomic locus of eukaryotic cells or prokaryotic cells.

As used herein, the term “RNA-guided nuclease” or “RGN” refers to a polypeptide that binds to a particular target nucleotide sequence in a nucleic acid molecule in a sequence-specific manner and is directed to the target sequence by a guide RNA molecule that is complexed with the polypeptide and hybridizes with the non-target strand of the target sequence. Although an RNA-guided nuclease can be capable of cleaving the target sequence upon binding, the term RNA-guided nuclease also encompasses nuclease-dead RNA-guided nucleases or RNA-guided nucleic acid binding proteins that are capable of binding to, but not cleaving, a target sequence. Cleavage of a target sequence by an RNA-guided nuclease can result in a single- or double-stranded break. RNA-guided nucleases only capable of cleaving a single strand of a double-stranded nucleic acid molecule are referred to herein as nickases. Non-limiting examples of RGNs or variants thereof that can be used in the presently disclosed compositions and methods, along with corresponding crRNA sequences and tracrRNA sequences (if needed), are presented in Table 1 below.

TABLE 1
Non-limiting examples of RNA-guided nucleases
and corresponding crRNA repeat sequences,
tracrRNA sequences, and PAM sequences.

| | RGN SEQ | crRNA repeat | | |
|---|---|---|---|---|
| RGN Name | ID NO: | sequence | tracrRNA | PAM |
| APG05083.1 | 62 | 63 | 64 | 65 |
| APG07433.1 | 53 | 66 | 67 | 65 |
| APG07513.1 | 68 | 69 | 70 | 65 |
| APG08290.1 | 52 | 71 | 72 | 73 |
| APG05459.1 | 74 | 75 | 76 | 77 |
| APG04583.1 | 78 | 79 | 80 | 81 |
| APG01688.1 | 82 | 83 | 84 | 85 |
| APG00969 | 86 | 87 | 88 | 89 |
| APG03128 | 90 | 91 | 92 | 93 |
| APG09748 | 94 | 95 | 96 | 97 |
| APG00771 | 98 | 99 | 100 | 101 |
| APG02789 | 102 | 103 | 104 | 105 |
| APG09106 | 106 | 107 | 108 | 97 |
| APG05733.1 | 109 | 110 | 111 | 112 |
| APG06207.1 | 113 | 114 | 115 | 116 |
| APG01647.1 | 117 | 118 | 119 | 120 |
| APG08032.1 | 121 | 122 | 123 | 124 |
| APG05712.1 | 125 | 126 | 127 | 128 |
| APG01658.1 | 129 | 130 | 131 | 132 |
| APG06498.1 | 133 | 134 | 135 | 136 |
| APG09106.1 | 137 | 138 | 139 | 140 |
| APG09882.1 | 141 | 142 | 143 | 144 |
| APG02675.1 | 145 | 146 | 147 | 116 |
| APG01405.1 | 148 | 149 | 150 | 151 |
| APG06250.1 | 152 | 153 | 154 | 155 |
| APG06877.1 | 156 | 157 | 158 | 112 |
| APG09053.1 | 159 | 160 | 161 | 162 |
| APG04293.1 | 163 | 164 | 165 | 166 |
| APG01308.1 | 167 | 168 | 169 | 170 |
| APG06646.1 | 171 | 172 | 173 | 166 |
| APG09624 | 174 | 175 | 176 | ND |
| APG05405 | 177 | 178 | 179 | ND |
| APG06622 | 180 | 181 | 182 | 183 |
| APG02787 | 184 | 185 | 186 | 187 |
| APG06248 | 188 | 189 | 190 | 191 |
| APG06007 | 192 | 193 | 194 | 195 |
| APG02874 | 196 | 197 | 198 | 199 |
| APG03850 | 200 | 201 | 202 | 203 |
| APG07553 | 204 | 205 | 206 | 207 |
| APG03031 | 208 | 209 | 210 | 199 |
| APG09208 | 211 | 212 | 213 | 214 |
| APG05586 | 215 | 216 | 217 | 218 |
| APG08770 | 219 | 220 | 221 | 218 |
| APG08167 | 222 | 223 | 224 | 225 |
| APG01604 | 226 | 227 | 228 | 225 |
| APG03021 | 229 | 230 | 231 | 232 |
| APG06015 | 233 | 234 | 235 | 236 |
| APG09344 | 237 | 238 | 239 | 240 |
| APG07991 | 241 | 242 | 243 | 244 |
| APG01868 | 245 | 246 | 247 | 244 |
| APG02998 | 248 | 249 | 250 | 244 |
| APG09298 | 251 | 252 | 253 | 254 |
| APG06251 | 255 | 252 | 253 | 254 |
| APG03066 | 256 | 252 | 253 | 254 |
| APG01560 | 257 | 252 | 253 | 254 |
| APG02777 | 258 | 252 | 253 | 254 |
| APG05761 | 259 | 252 | 253 | 254 |
| APG02479 | 260 | 252 | 253 | 254 |
| APG08385 | 261 | 252 | 253 | 254 |
| APG09217 | 262 | 252 | 253 | 254 |
| APG06657 | 263 | 252 | 253 | 254 |
| APG05586 | 264 | 252 | 253 | 254 |
| APG07433.1 deletion | 265 | 266 | 267 | 268 |
| variant | | | | |
| APG08290.1 deletion | 269 | 270 | 271 | 272 |
| variant | | | | |

* ND: not determined

According to the present invention, a target nucleotide sequence is bound by an RNA-guided nuclease. The non-target strand of the target DNA sequence hybridizes with the guide RNA associated with the RNA-guided nuclease. The target strand and/or the non-target strand of the target DNA sequence can then be subsequently cleaved by the RNA-guided nuclease if the polypeptide possesses nuclease activity. The terms “cleave” or “cleavage” refer to the hydrolysis of at least one phosphodiester bond within the backbone of one or both strands of a target DNA sequence that can result in either single-stranded or double-stranded breaks within the target DNA sequence. RGNs can cleave nucleotides within a polynucleotide, functioning as an endonuclease or can be an exonuclease, removing successive nucleotides from the end (the 5′ and/or the 3′ end) of a polynucleotide. In other embodiments, the disclosed RGNs can cleave nucleotides of a target polynucleotide within any position of a polynucleotide and thus function as both an endonuclease and exonuclease. The cleavage of a target polynucleotide by RGNs can result in staggered breaks or blunt ends.

The RNA-guided nucleases can be wild-type sequences derived from bacterial or archaeal species. Alternatively, the RNA-guided nucleases can be variants or fragments of wild-type polypeptides. The wild-type RGN can be modified to alter nuclease activity or alter PAM specificity, for example. In some embodiments, the RNA-guided nuclease is not naturally-occurring.

In some embodiments, the RNA-guided nuclease functions as a nickase, only cleaving a single strand of the target DNA sequence. Such RNA-guided nucleases have a single functioning nuclease domain. In some embodiments, the nickase is capable of cleaving the target strand or the non-target strand of the target DNA sequence. In some of these embodiments, additional nuclease domains have been mutated such that the nuclease activity is reduced or eliminated. In embodiments wherein a nickase is used, in order to effect a double-stranded cleavage of a target DNA sequence, two nickases are needed, each of which nicks a single strand within the target DNA sequence.

In other embodiments, the RNA-guided nuclease lacks nuclease activity altogether, and is referred to herein as nuclease-dead or nuclease inactive. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety.

RNA-guided nucleases that lack nuclease activity can be used to deliver a fused polypeptide, polynucleotide, or small molecule payload to a particular genomic location. In some of these embodiments, the RGN polypeptide or guide RNA can be fused to a detectable label to allow for detection of a particular sequence. As a non-limiting example, a nuclease-dead RGN can be fused to a detectable label (e.g., fluorescent protein) and targeted to a particular sequence associated with a disease to allow for detection of the disease-associated sequence.

Alternatively, nuclease-dead RGNs can be targeted to particular genomic locations to alter the expression of a desired gene. In some embodiments, the binding of a nuclease-dead RNA-guided nuclease to a target sequence within a target gene results in the reduction in expression of the target or a gene under transcriptional control by the target sequence by interfering with the binding of RNA polymerase or transcription factors within the targeted genomic region. In other embodiments, the RGN (e.g., a nuclease-dead RGN) or its complexed guide RNA further comprises an expression modulator that, upon binding to a target sequence, serves to either repress or activate the expression of the target gene or a gene under transcriptional control by the target sequence. In some of these embodiments, the expression modulator modulates the expression of the target gene or regulated gene through epigenetic mechanisms.

In other embodiments, the nuclease-dead RGNs or an RGN with nickase activity can be targeted to particular genomic locations to modify the sequence of a target polynucleotide through fusion to a base-editing polypeptide, for example a deaminase polypeptide or active variant or fragment thereof, that directly chemically modifies (e.g., deaminates) a nucleobase, resulting in conversion from one nucleobase to another. The base-editing polypeptide can be fused to the RGN at its N-terminal or C-terminal end. Additionally, the base-editing polypeptide may be fused to the RGN via a peptide linker. A non-limiting example of a deaminase polypeptide that is useful for such compositions and methods includes a cytosine deaminase or an adenine deaminase (such as the adenine deaminase base editor described in Gaudelli et al. (2017) Nature 551:464-471, U.S. Publ. Nos. 2017/0121693 and 2018/0073012, and International Publ. No. WO 2018/027078, or any of the deaminases disclosed in International Publ. Nos. WO 2020/139783 and WO 2022/056254, and International Application No. PCT/US2022/21271 filed Mar. 22, 2022, each of which is herein incorporated by reference in its entirety). Further, it is known in the art that certain fusion proteins between an RGN and a base-editing enzyme (e.g., cytosine deaminase) may also comprise at least one uracil stabilizing polypeptide that increases the mutation rate of a cytidine, deoxycytidine, or cytosine to a thymidine, deoxythymidine, or thymine in a nucleic acid molecule by a deaminase. Non-limiting examples of uracil stabilizing polypeptides include those disclosed in International Publ. No. WO 2021/217002, which is herein incorporated by reference in its entirety, including USP2 (SEQ ID NO: 59) and a uracil glycosylase inhibitor (UGI) domain (SEQ ID NO: 58), which may increase base editing efficiency. Therefore, a fusion protein may comprise an RGN, a deaminase, and optionally at least one uracil stabilizing polypeptide, such as UGI or USP2. In some embodiments, the RGN that is fused to the base-editing polypeptide is a nickase that cleaves the DNA strand that is not acted upon by the base-editing polypeptide (e.g., deaminase).

RNA-guided nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RNA guided nuclease and a base-editing polypeptide, such as a deaminase. In some embodiments, a linker joins a nuclease-dead RGN and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5,6,7, 8,9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

RNA-guided nucleases can comprise at least one nuclear localization signal (NLS) to enhance transport of the RGN to the nucleus of a cell. Nuclear localization signals are known in the art and generally comprise a stretch of basic amino acids (see, e.g., Lange et al., J Biol. Chem. (2007) 282:5101-5105). In some embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleoplasmin, and c-Myc (see, e.g., Ray et al. (2015) Bioconjug Chem 26(6):1004-7). In some embodiments, the RGN comprises the NLS sequence set forth as SEQ ID NO: 60 and/or 61. The RGN can comprise one or more NLS sequences at its N-terminus, C-terminus, or both the N-terminus and C-terminus. For example, the RGN can comprise two NLS sequences at the N-terminal region and four NLS sequences at the C-terminal region.

Other localization signal sequences known in the art that localize polypeptides to particular subcellular location(s) can also be used to target the RGNs, including, but not limited to, plastid localization sequences, mitochondrial localization sequences, and dual-targeting signal sequences that target to both the plastid and mitochondria (see, e.g., Nassoury and Morse (2005) Biochim Biophys Acta 1743:5-19; Kunze and Berger (2015) Front Physiol dx.doi.org/10.3389/fphys.2015.00259; Herrmann and Neupert (2003) IUBMB Life 55:219-225; Soll (2002) Curr Opin Plant Biol 5:529-535; Carrie and Small (2013) Biochim BiophysActa 1833:253-259; Carrie et al. (2009) FEBSJ276:1187-1195; Silva-Filho (2003) Curr Opin Plant Biol 6:589-595; Peeters and Small (2001) Biochim Biophys Acta 1541:54-63; Murcha et al. (2014) J Exp Bot 65:6301-6335; Mackenzie (2005) Trends Cell Biol 15:548-554; Glaser et al. (1998) Plant Mol Biol 38:311-338).

In some embodiments, the RNA-guided nuclease comprises at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the RNA-guided nuclease.

The RGN can be fused to an effector domain, such as a cleavage domain, a deaminase, or an expression modulator domain, either directly or indirectly via a linker peptide. Such a domain can be located at the N-terminus, the C-terminus, or an internal location of the RNA-guided nuclease. In some of these embodiments, the RGN component of the fusion protein is a nuclease-dead RGN or a nickase.

In some embodiments, the RGN fusion protein comprises a cleavage domain, which is any domain that is capable of cleaving a polynucleotide (i.e., RNA, DNA, or RNA/DNA hybrid) and includes, but is not limited to, restriction endonucleases and homing endonucleases, such as Type IIS endonucleases (e.g., FokI) (see, e.g., Belfort et al. (1997) Nucleic Acids Res. 25:3379-3388; Linn et al. (eds.) Nucleases, Cold Spring Harbor Laboratory Press, 1993).

In other embodiments, the RGN fusion protein comprises a deaminase that deaminates a nucleobase, resulting in conversion from one nucleobase to another, and includes, but is not limited to, a cytosine deaminase or an adenine deaminase (see, e.g., Gaudelli et al. (2017) Nature 551:464-471, U.S. Publ. Nos. 2017/0121693 and 2018/0073012, U.S. Pat. No. 9,840,699, International Publ. No. WO 2018/027078, and International Publ. No. WO 2018/027078, or any of the deaminases disclosed in International Publ. Nos. WO 2020/139783 and WO 2022/056254, and International Appl. No. PCT/US2022/021271 filed Mar. 22, 2022, each of which is herein incorporated by reference in its entirety).

In some embodiments, the effector domain of the RGN fusion protein can be an expression modulator domain, which is a domain that either serves to upregulate or downregulate transcription. The expression modulator domain can be an epigenetic modification domain, a transcriptional repressor domain or a transcriptional activation domain.

In some of these embodiments, the expression modulator of the RGN fusion protein comprises an epigenetic modification domain that covalently modifies DNA or histone proteins to alter histone structure and/or chromosomal structure without altering the DNA sequence, leading to changes in gene expression (i.e., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modification domains include histone acetyltransferase domains, histone deacetylase domains, histone methyltransferase domains, histone demethylase domains, DNA methyltransferase domains, and DNA demethylase domains.

In other embodiments, the expression modulator of the fusion protein comprises a transcriptional repressor domain, which interacts with transcriptional control elements and/or transcriptional regulatory proteins, such as RNA polymerases and transcription factors, to reduce or terminate transcription of at least one gene. Transcriptional repressor domains are known in the art and include, but are not limited to, Sp1-like repressors, IκB, and Krüppel associated box (KRAB) domains.

In yet other embodiments, the expression modulator of the fusion protein comprises a transcriptional activation domain, which interacts with transcriptional control elements and/or transcriptional regulatory proteins, such as RNA polymerases and transcription factors, to increase or activate transcription of at least one gene. Transcriptional activation domains are known in the art and include, but are not limited to, a herpes simplex virus VP16 activation domain and an NFAT activation domain.

The RGN polypeptide can comprise a detectable label or a purification tag. The detectable label or purification tag can be located at the N-terminus, the C-terminus, or an internal location of the RNA-guided nuclease, either directly or indirectly via a linker peptide. In some of these embodiments, the RGN component of the fusion protein is a nuclease-dead RGN. In other embodiments, the RGN component of the fusion protein is an RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

RGN polypeptides can also comprise a purification tag, which is any molecule that can be utilized to isolate a protein or fused protein from a mixture (e.g., biological sample, culture medium). Non-limiting examples of purification tags include biotin, myc, maltose binding protein (MBP), glutathione-S-transferase (GST), and 3×FLAG tag.

### VI. Variants and Fragments of Polypeptides and Polynucleotides

The present disclosure provides active variants and fragments of a naturally-occurring (i.e., wild-type) promoter, the nucleotide sequence of which is set forth as SEQ ID NOs: 1-5, as well as active variants and fragments of a truncated version of these wild-type sequences. The nucleotide sequences of the truncated promoters are set forth as SEQ ID NOs: 6-10 and the present disclose contemplates active variants and fragments of these truncated promoters.

While the activity of a variant or fragment may be altered compared to the polynucleotide of interest (e.g., promoter) or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of any one of SEQ ID NOs: 1-10 will retain transcription initiation activity of an operably linked coding sequence. In some embodiments, fragments and variants of any one of SEQ ID NOs: 1-10 will retain the ability to recruit pol III and initiate transcription therefrom. In some embodiments, fragments and variants of any one of SEQ ID NOs: 1-10 will retain the ability to recruit pol II and initiate transcription therefrom. In some embodiments, active variants or fragments of the presently disclosed promoters comprise a TATA box sequence, an OCT sequence, a SPH sequence, and/or a PSE sequence. In some embodiments, active variants or fragments of any one of SEQ ID NOs: 1-5 will lack the putative nucleosomal sequence (e.g., the internal sequence that has been removed from SEQ ID NOs: 1-5 to generate SEQ ID NOs: 6-10, respectively).

Fragments and variants of naturally-occurring RGN polypeptides, such as those disclosed herein in Table 1, will retain sequence-specific, RNA-guided nucleic acid-binding activity. In some embodiments, fragments and variants of naturally-occurring RGN polypeptides, such as those disclosed herein in Table 1, will retain nuclease activity (single-stranded or double-stranded).

Fragments and variants of naturally-occurring CRISPR repeats, such as those disclosed herein in Table 1, will retain the ability, when part of a guide RNA (comprising a tracrRNA), to bind to and guide an RNA-guided nuclease (complexed with the guide RNA) to a target nucleotide sequence in a sequence-specific manner.

Fragments and variants of naturally-occurring tracrRNAs, such as those disclosed herein in Table 1, will retain the ability, when part of a guide RNA (comprising a CRISPR RNA), to guide an RNA-guided nuclease (complexed with the guide RNA) to a target nucleotide sequence in a sequence-specific manner.

The term “fragment” refers to a portion of a polynucleotide or polypeptide sequence of the invention. “Fragments” or “biologically active portions” include polynucleotides comprising a sufficient number of contiguous nucleotides to retain the biological activity (e.g., initiating transcription). In some embodiments, two or more fragments can be combined to generate an active variant. “Fragments” or “biologically active portions” include polypeptides comprising a sufficient number of contiguous amino acid residues to retain the biological activity (e.g., binding to a target nucleotide sequence in a sequence-specific manner when complexed with a guide RNA). Fragments of RGN proteins include those that are shorter than the full-length sequences due to the use of an alternate downstream start site. A biologically active fragment of a promoter can be a polynucleotide that comprises at least 10 contiguous nucleotides of any one of SEQ ID NOs: 1-10, including but not limited to, about 10 to about 290, about 20 to about 290, about 30 to about 290, about 40 to about 290, about 45 to about 290, about 50 to about 290, about 55 to about 290, about 60 to about 290, about 65 to about 290, about 67 to about 290, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 120, 125, 130, 135, 140, 145, 150, 160, 170, 180, 190,200, 210, 220, 230, 240, 250, 260, 270, 280, 290, or 300 contiguous nucleotides of any one of SEQ ID NOs: 1-10.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 30%, 31%, 32%, 33%, 34%, 35%, 36%, 37%, 38%, 39%, 40%, 45%, 50%, 55%, 60%, 65%, 70%, 75%, 80%, 85%, 90%, 91%, 92%, 93%, 94%, 95%, 96%, 97%, 98%, 99% or more sequence identity.

A biologically active variant of a promoter of the invention may differ by as few as about 1-15 nucleotides, as few as about 1-10, such as about 6-10, as few as 5, as few as 4, as few as 3, as few as 2, or as few as 1 nucleotide. In specific embodiments, the promoter can comprise a 5′ or 3′ truncation, which can comprise at least a deletion of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 70, 75, 80, 85, 90, 95, 100, 150, 200, 250, nucleotides or more from either the 5′ or 3′ end of the polynucleotide.

In some embodiments, the presently disclosed promoters comprise a nucleotide sequence having at least 30%, 31%, 32%, 33%, 34%, 35%, 36%, 37%, 38%, 39%, 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as any one of SEQ ID NOs: 1-10.

It is recognized that modifications may be made to the promoters provided herein, as well as the RGN polypeptides, CRISPR repeats, and tracrRNAs provided herein in Table 1, creating variant proteins and polynucleotides. Changes designed by man may be introduced through the application of site-directed mutagenesis techniques. Alternatively, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter or improve the function of the RGN proteins listed in Table 1.

Variant polynucleotides and proteins also encompass sequences and proteins derived from a mutagenic and recombinogenic procedure such as DNA shuffling. With such a procedure, one or more different sequences is manipulated to create a new sequence possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VII. Methods for Expressing an RNA or Polypeptide

The presently disclosed promoters (i.e., SEQ ID NOs: 1-10 or an active variant or fragment of any thereof) can be used to express an RNA or polypeptide through operable linkage with a coding sequence. Methods for preparing such an expression construct and methods for expressing an RNA or polypeptide are therefore provided. Methods for making an RGN ribonucleotide complex and purifying the same are further provided.

Methods for preparing an expression construct comprise inserting a coding sequence into a nucleic acid molecule comprising a presently disclosed promoter or inserting a presently disclosed promoter into a nucleic acid molecule comprising a coding sequence such that the coding sequence is operably linked to the promoter. Any method known in the art to insert a nucleotide sequence (e.g., coding sequence or promoter) into a nucleic acid molecule can be used. The expression construct can be prepared or generated in vitro or in vivo. A non-limiting example of in vitro expression construct preparation involves cleavage of the nucleic acid molecule with a restriction endonuclease, for example, to generate blunt ends or overhangs (5′ or 3′) that are compatible with the overhangs on the sequence to be inserted, followed by ligation. A non-limiting example of a method for preparing an expression construct in vivo is to use a gene editing approach (e.g., zinc finger nuclease, TALEN, RGN) to generate blunt or staggered ends in a nucleic acid molecule and a donor polynucleotide with blunt ends or compatible overhangs that can be inserted into the cleavage site.

In vitro or in vivo methods can also be used for expressing an RNA or polypeptide. In vitro methods for expressing an RNA comprise contacting an expression construct comprising a presently disclosed promoter operably linked to an RNA-coding sequence with an RNA polymerase III and ribonucleotide triphosphates (also referred to as rNTPs) (i.e., rATP, rUTP, rCTP, and rGTP). In vitro methods for expression of a polypeptide comprise first making an mRNA in vitro by contacting an expression construction comprising a presently disclosed promoter operably linked to a polypeptide-coding sequence with an RNA polymerase II and (rNTPs). In some embodiments, a cap analog is also present in the reaction mix to generate a capped mRNA. In some embodiments, the method further comprises contacting the capped mRNA with a poly(A) polyermase to generate a capped and tailed mRNA. The capped and tailed mRNA can then be contacted in vitro with a cell-free lysate, such as wheat germ extract, rabbit reticulocyte extracts, or E. coli extracts, or purified components to generate an in vitro translated polypeptide. In some embodiments, in vitro transcription and translation are performed in a single-step process wherein the expression construct is contacted with all of the components needed for both in vitro transcription and translation.

In vivo methods for expressing an RNA or polypeptide comprise introducing into a cell an expression construct comprising a presently disclosed promoter operably linked to a coding sequence. The coding sequence can be for an RNA or a polypeptide.

By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. The host cell can be a eukaryotic or prokaryotic cell. In some embodiments, the eukaryotic host cell is a plant cell, a mammalian cell, an avian cell, or an insect cell. In some embodiments, the eukaryotic cell is a human cell. In some embodiments, the eukaryotic cell is a cell of hematopoietic origin, such as an immune cell (i.e., a cell of the innate or adaptive immune system), including but not limited to a B cell, a T cell, a natural killer (NK) cell, a pluripotent stem cell, an induced pluripotent stem cell, a chimeric antigen receptor T (CAR-T) cell, a monocyte, a macrophage, and a dendritic cell.

Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a promoter of the invention, and in some embodiments, an operably linked coding sequence. It is recognized that other exogenous or endogenous nucleic acid sequences or DNA fragments may also be incorporated into the host cell. Agrobacterium- and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, crRNA, and/or tracrRNA includes retroviral, lentiviral, adenoviral, and adeno-associated viral mediated introduction and expression, as well as the use of Caulimoviruses, Geminiviruses, and RNA plant viruses.

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5.

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Nail. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Alternatively, cells that have been transformed may be introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. In some embodiments, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

Polynucleotides comprising the presently disclosed promoters, and in some embodiments, and operably linked coding sequence can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

Polynucleotides comprising the presently disclosed promoters, and in some embodiments, and operably linked coding sequence can be used to transform any eukaryotic species, including but not limited to animals (e.g., mammals, insects, fish, birds, and reptiles), fungi, amoeba, algae, and yeast.

Conventional viral and non-viral based gene transfer methods can be used to introduce nucleic acids in mammalian, insect, or avian cells or target tissues. Such methods can be used to administer nucleic acids to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256: 808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., 1. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., 1. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In some embodiments, the cell line may be mammalian, insect, or avian cells. A wide variety of cell lines for tissue culture are known in the art. Examples of cell lines include, but are not limited to, C8161, CCRF-CEM, MOLT, mIMCD-3, NHDF, HeLaS3, Huh1, Huh4, Huh7, HUVEC, HASMC, HEKn, HEKa, MiaPaCell, Panel, PC-3, TFl, CTLL-2, CIR, Rat6, CVI, RPTE, AlO, T24, 182, A375, ARH-77, Calul, SW480, SW620, SKOV3, SK-UT, CaCo2, P388D1, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L235010, CORL23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, lY cells, K562 cells, Ku812, KCL22, KGl, KYOl, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-IA, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-IA/PNT 2, RenCa, RIN-5F, RMA/RMAS, Saos-2 cells, Sf-9, SkBr3, T2, T-47D, T84, THPl cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with a polynucleotide comprising a presently disclosed promoter, and in some embodiments, an operably linked coding sequence (such as by transient transfection of one or more vectors, or transfection with RNA), or modified through the activity of an RGN system, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, hamster, rabbit, cow, or pig. In some embodiments, the transgenic animal is a bird, such as a chicken or a duck. In some embodiments, the transgenic animal is an insect, such as a mosquito or a tick.

Methods for making an RGN ribonucleoprotein complex are also provided. These methods can comprise introducing an expression construct comprising a presently disclosed promoter (i.e., SEQ ID NOs: 1-10 or an active variant or fragment of any thereof) operably linked to a guide RNA-encoding sequence into a cell that comprises an RGN. In some embodiments, methods for making an RGN ribonucleoprotein complex comprise introducing into a cell a first expression construct comprising a pol III promoter (such as a presently disclosed promoter) operably linked to a guide RNA-encoding sequence and a second expression construct comprising a pol II promoter (such as a presently disclosed promoter) operably linked to an RGN-coding sequence. In some of these embodiments, the first and second expression constructs are located on a single vector. In other embodiments, the first and second expression cassettes are located on separate vectors. In some embodiments, methods for making an RGN ribonucleoprotein complex comprise introducing into a cell: (i) a chemically synthesized guide RNA or a chemically synthesized polynucleotide encoding the guide RNA; and (ii) an expression construct comprising a pol II promoter (such as a presently disclosed promoter) operably linked to an RGN-coding sequence. In some embodiments, methods for making an RGN ribonucleoprotein complex comprise introducing into a cell: (i) an expression construct comprising a pol III promoter (such as a presently disclosed promoter) operably linked to a guide RNA-encoding sequence and a polynucleotide encoding an RGN. In some of these embodiments, the polynucleotide encoding an RGN is an mRNA molecule. In some embodiments, an mRNA encoding an RGN useful in the presently disclosed methods and compositions can include one or more structural and/or chemical modifications or alterations which impart useful properties to the mRNA. For instance, a useful property of an mRNA includes the lack of a substantial induction of the innate immune response of a cell into which the mRNA is introduced. A “structural” feature or modification is one in which two or more linked nucleotides are inserted, deleted, duplicated, inverted or randomized in an mRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. Chemical modifications to mRNA can involve inclusion of 5-methylcytosine, N1-methyl-pseudouridine, pseudouridine, 2-thiouridine, 4-thiouridine, 5-methoxyuridine, 2′Fluoroguanosine, 2′Fluorouridine, 5-bromouridine, 5-(2-carbomethoxyvinyl) uridine, 5-[3(1-E-propenylamino)] uridine, α-thiocytidine, N6-methyladenosine, 5-methylcytidine, N4-acetylcytidine, 5-formylcytidine, or combinations thereof, in an mRNA. The RGN ribonucleoprotein complex can then be purified from a lysate of the cells.

Methods for purifying an RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, or ribonucleoprotein complex, is substantially or essentially free from components that normally accompany or interact with the polypeptide as found in its naturally occurring environment. Thus, an isolated or purified polypeptide or ribonucleoprotein complex is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals. Similarly, an “isolated” polynucleotide or nucleic acid molecule is removed from its naturally occurring environment. An isolated polynucleotide is substantially free of chemical precursors or other chemicals when chemically synthesized or has been removed from a genomic locus via the breaking of phosphodiester bonds. An isolated polynucleotide can be part of a vector, a composition of matter or can be contained within a cell so long as the cell is not the original environment of the polynucleotide.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

A purified or in vitro assembled RGN ribonucleoprotein complex can be introduced into a cell, organelle, or embryo using any method known in the art, including, but not limited to electroporation. Alternatively, an RGN polypeptide and/or polynucleotide encoding or comprising the guide RNA can be introduced into a cell, organelle, or embryo using any method known in the art (e.g., electroporation).

### VII. Methods of Binding, Cleaving, or Modifying a Target Nucleic Acid Molecule

The present disclosure provides methods for binding, cleaving, and/or modifying a target nucleic acid molecule comprising a target sequence of interest. The methods include introducing into a cell one or more polynucleotides comprising a first expression construct comprising a presently disclosed promoter operably linked to a guide RNA, crRNA, and/or tracrRNA and a second expression construct comprising an RGN-coding sequence. In some embodiments, the method comprises introducing into a cell a polynucleotide comprising an expression construct comprising a presently disclosed promoter operably linked to a guide RNA, wherein the cell comprises an RGN that is capable of binding the introduced guide RNA, thereby directing the RGN to a target sequence. In some embodiments, the method comprises introducing into a cell a polynucleotide comprising an expression construct comprising a presently disclosed promoter operably linked to a crRNA, wherein the cell comprises an RGN and tracrRNA that is capable of binding the introduced crRNA, thereby directing the RGN to a target sequence. The RGN may be a nuclease dead RGN, have nickase activity, or may be a fusion polypeptide. In some embodiments, the fusion polypeptide comprises a base-editing polypeptide, for example a cytosine deaminase or an adenine deaminase. In other embodiments, the RGN fusion protein comprises a reverse transcriptase. In other embodiments, the RGN fusion protein comprises a polypeptide that recruits members of a functional nucleic acid repair complex, such as a member of the nucleotide excision repair (NER) or transcription coupled-nucleotide excision repair (TC-NER) pathway (Wei et al., 2015, PNAS USA 112(27):E3495-504; Troelstra et al., 1992, Cell 71:939-953; Marnef et al., 2017, J Mol Biol 429(9):1277-1288), as described in U.S. Provisional Application No. 63/332,486, which was filed on Apr. 19, 2022, and is incorporated by reference in its entirety. In some embodiments, the RGN fusion protein comprises CSB (van den Boom et al., 2004, J Cell Biol 166(1):27-36; van Gool et al., 1997, EMBO J 16(19):5955-65), which is a member of the TC-NER (nucleotide excision repair) pathway and functions in the recruitment of other members. In further embodiments, the RGN fusion protein comprises an active domain of CSB, such as the acidic domain of CSB.

In some embodiments, the RGN polypeptide, guide RNA and/or crRNA is heterologous to the cell, organelle, or embryo to which the polynucleotide encoding the RGN polypeptide, guide RNA, crRNA, and/or tracrRNA is introduced.

In those embodiments wherein the method comprises delivering a polynucleotide encoding a guide RNA, crRNA, tracrRNA, and/or an RGN polypeptide, the cell or embryo can then be cultured under conditions in which the guide RNA, crRNA, and/or RGN polypeptide are expressed. In some embodiments, the method comprises contacting a target sequence with an RGN ribonucleoprotein complex. The RGN ribonucleoprotein complex may comprise an RGN that is nuclease dead or has nickase activity. In some embodiments, the RGN of the ribonucleoprotein complex is a fusion polypeptide comprising a base-editing polypeptide. In some embodiments, the method comprises introducing into a cell, organelle, or embryo comprising a target sequence an RGN ribonucleoprotein complex. The RGN ribonucleoprotein complex can be one that has been purified from a biological sample, recombinantly produced and subsequently purified, or in vitro-assembled as described herein. In those embodiments wherein the RGN ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled RGN ribonucleoprotein complex can be introduced into a cell, organelle, or embryo using any method known in the art, including, but not limited to electroporation. Alternatively, an RGN polypeptide and/or polynucleotide encoding or comprising the guide RNA can be introduced into a cell, organelle, or embryo using any method known in the art (e.g., electroporation).

Upon delivery to or contact with the target sequence or cell, organelle, or embryo comprising the target sequence, the guide RNA directs the RGN to bind to the target sequence in a sequence-specific manner. In those embodiments wherein the RGN has nuclease activity, the RGN polypeptide cleaves the target sequence of interest upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of a single type of RGN complexed with more than one guide RNA. The more than one guide RNA can target different regions of a single gene or can target multiple genes.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a modification to the target sequence. As used herein, a “modification” in reference to a nucleic acid molecule refers to a change in the nucleotide sequence of the nucleic acid molecule, which can be a deletion, insertion, or substitution of one or more nucleotides, or a combination thereof. Modification of the target sequence can result in the expression of an altered protein product or inactivation of a coding sequence.

In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest. In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences, referred to herein as “homology arms” that have substantial sequence identity with the sequences flanking the target sequence, allowing for a homology-directed repair process. In some embodiments, homology arms have a length of at least 50 base pairs, at least 100 base pairs, and up to 2000 base pairs or more, and have at least 90%, at least 95%, or more, sequence homology to their corresponding sequence within the target nucleotide sequence.

In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target loci and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide.

In some embodiments, a method is provided for binding a target nucleotide sequence and detecting the target sequence, wherein the method comprises introducing into a cell, organelle, or embryo one or more polynucleotides comprising a first expression construct comprising a presently disclosed promoter operably linked to a coding sequence for a guide RNA, and a second expression construct comprising a coding sequence for an RGN polypeptide, wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a target gene of interest comprising a target sequence or a gene under the regulation of a target sequence. The methods comprise introducing into a cell, organelle, or embryo one or more polynucleotides comprising a first expression construct comprising a presently disclosed promoter operably linked to a coding sequence for a guide RNA, crRNA, and/or tracrRNA, and a second expression construct comprising a coding sequence for an RGN polypeptide, wherein the RGN polypeptide is a nuclease-dead RGN. In some of these embodiments, the nuclease-dead RGN is a fusion protein comprising an expression modulator domain (i.e., epigenetic modification domain, transcriptional activation domain or a transcriptional repressor domain) as described herein.

The present disclosure also provides methods for binding and/or modifying a target nucleic acid molecule of interest comprising a target sequence. The methods include introducing into a cell, organelle, or embryo one or more polynucleotides comprising a first expression construct comprising a presently disclosed promoter operably linked to a coding sequence for a guide RNA, crRNA, and/or tracrRNA, and a second expression construct comprising a coding sequence for a fusion polypeptide comprising an RGN and a base-editing polypeptide, for example a cytosine deaminase or an adenine deaminase. In some of these embodiments, the RGN of the fusion polypeptide is a nickase or a nuclease-dead RGN.

In some embodiments wherein a fusion polypeptide comprising an RGN and a base-editing polypeptide is utilized, the binding of the fusion protein to a target sequence results in the modification of nucleotide(s) adjacent to the target sequence. The nucleobase adjacent to the target sequence that is modified by the deaminase may be 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs from the 5′ or 3′ end of the target sequence.

One of ordinary skill in the art will appreciate that any of the presently disclosed methods can be used to target a single target sequence or multiple target sequences. Thus, methods comprise the use of a single RGN polypeptide in combination with multiple, distinct guide RNAs, which can target multiple, distinct sequences within a single gene and/or multiple genes. Also encompassed herein are methods wherein multiple, distinct guide RNAs are introduced in combination with multiple, distinct RGN polypeptides. These guide RNAs and guide RNA/RGN polypeptide systems can target multiple, distinct sequences within a single gene and/or multiple genes.

Methods for reducing the expression of a target gene in a cell can also comprise introducing into the cell an expression construct comprising a presently disclosed promoter operably linked to a coding sequence for a gene silencing RNA (e.g., shRNA, siRNA, antisense RNA, or the like), wherein the gene silencing RNA reduces the expression of the target gene.

In one aspect, the invention provides kits containing any one or more of the elements disclosed in the above methods and compositions. In some embodiments, the kit comprises a vector system and instructions for using the kit. In some embodiments, the vector system comprises (a) an expression construct comprising a presently disclosed promoter operably linked to a coding sequence for a guide RNA, wherein the spacer sequence is absent and one or more insertion sites for inserting a spacer sequence are present; and, in some embodiments, (b) a second expression construct comprising an RGN-coding sequence. Elements may be provided individually or in combinations, and may be provided in any suitable container, such as a vial, a bottle, or a tube.

In some embodiments, the kit includes instructions in one or more languages. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises a homologous recombination donor template polynucleotide.

### IX. Target Polynucleotides

In one aspect, the invention provides for methods of modifying a target polynucleotide comprising a target sequence or modifying the expression of a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be re-introduced into the non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can analyze the genome for sources of useful genes, and in varieties having desired characteristics or traits employ the present invention to induce the rise of useful genes, with more precision than previous mutagenic agents and hence accelerate and improve plant breeding programs.

The target polynucleotide of an RGN or gene silencing RNA can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, the target strand of the target sequence should be associated with a PAM (protospacer adjacent motif); that is, a short sequence recognized by the RGN. The precise sequence and length requirements for the PAM differ depending on the RGN used, but PAMs are typically 2-7 base pair sequences adjacent to the protospacer (that is, the target strand of the target sequence).

The target polynucleotide of an RGN or gene silencing RNA may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level. In some embodiments, the disease may be an animal disease. In some embodiments, the disease may be an avian disease. In other embodiments, the disease may be a mammalian disease. In further embodiments, the disease may be a human disease. Examples of disease-associated genes and polynucleotides in humans are available from McKusick-Nathans Institute of Genetic Medicine, Johns Hopkins University (Baltimore, Md.) and National Center for Biotechnology Information, National Library of Medicine (Bethesda, Md.), available on the World Wide Web.

Although RGNs are particularly useful for their relative ease in targeting to genomic sequences of interest, there still remains an issue of what the RGN can do to address a causal mutation. One approach is to produce a fusion protein between an RGN (e.g., an inactive or nickase variant of the RGN) and a base-editing enzyme or the active domain of a base editing enzyme, such as a cytosine deaminase or an adenine deaminase base editor (U.S. Pat. No. 9,840,699, herein incorporated by reference). In some embodiments, the methods comprise contacting a DNA molecule comprising a target sequence with (a) a fusion protein comprising an RGN of the disclosure or a nickase variant thereof and a base-editing polypeptide such as a deaminase; and (b) a gRNA targeting the fusion protein of (a) to the target sequence; wherein the DNA molecule is contacted with the fusion protein and the gRNA in an amount effective and under conditions suitable for the deamination of a nucleobase. In some embodiments, the target DNA sequence comprises a sequence associated with a disease or disorder, and wherein the deamination of the nucleobase results in a sequence that is not associated with a disease or disorder. In some embodiments, the target DNA sequence resides in an allele of a crop plant, wherein the particular allele of the trait of interest results in a plant of lesser agronomic value. The deamination of the nucleobase results in an allele that improves the trait and increases the agronomic value of the plant.

In some embodiments, the target DNA sequence comprises a T→C or A→G point mutation associated with a disease or disorder, and wherein the deamination of the mutant C or G base results in a sequence that is not associated with a disease or disorder. In some embodiments, the deamination corrects a point mutation in the sequence associated with the disease or disorder.

In some embodiments, the sequence associated with the disease or disorder encodes a protein, and wherein the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

### X. Pharmaceutical Compositions and Methods of Treatment

Pharmaceutical compositions comprising one or more polynucleotides comprising an expression construct comprising a presently disclosed promoter (i.e., SEQ ID NOs: 1-10 or an active variant or fragment of any thereof) operably linked to a coding sequence, such as one encoding a gene silencing RNA, a guide RNA, a crRNA, or a polypeptide, and a pharmaceutically acceptable carrier are provided. In some of those embodiments wherein the presently disclosed promoter is operably linked to a coding sequence for a guide RNA or a crRNA, the pharmaceutical composition further comprises an RGN-coding sequence, and in some embodiments, a promoter operably linked thereto. In some of these embodiments, the promoter operably linked to the RGN-coding sequence is a presently disclosed promoter.

A pharmaceutical composition is a composition that is employed to prevent, reduce in intensity, cure or otherwise treat a target condition or disease that comprises an active ingredient (i.e., an expression construct comprising a presently disclosed promoter operably linked to a coding sequence, or cells comprising the expression construct) and a pharmaceutically acceptable carrier.

As used herein, a “pharmaceutically acceptable carrier” refers to a material that does not cause significant irritation to an organism and does not abrogate the activity and properties of the active ingredient (i.e., an expression construct comprising a presently disclosed promoter operably linked to a coding sequence, or cells comprising the expression construct). Carriers must be of sufficiently high purity and of sufficiently low toxicity to render them suitable for administration to a subject being treated. The carrier can be inert, or it can possess pharmaceutical benefits. In some embodiments, a pharmaceutically acceptable carrier comprises one or more compatible solid or liquid filler, diluents or encapsulating substances which are suitable for administration to a human or other vertebrate animal. In some embodiments, the pharmaceutically acceptable carrier is not naturally-occurring. In some embodiments, the pharmaceutically acceptable carrier and the active ingredient are not found together in nature.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. See, e.g., Remington, The Science and Practice of Pharmacy (21st ed. 2005). Suitable formulations include, for example, powders, pastes, ointments, jellies, waxes, oils, lipids, lipid (cationic or anionic) containing vesicles (such as LIPOFECTIN vesicles), liposomes, lipid nanoparticles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc.

The disclosure provides for pharmaceutical compositions comprising lipid-based formulations including an active ingredient (i.e. guide RNAs and/or RGNs, or polynucleotides comprising or encoding such). In some embodiments, the lipid-based formulations include liposomes. In some embodiments, the lipid-based formulations include lipid nanoparticles (LNPs). In some embodiments, an active ingredient is encapsulated in the lipid particle and/or disposed on the surface of the lipid particle. In some embodiments, an active ingredient is covalently attached to the lipid particle. In some embodiments, an active ingredient is non-covalently associated with the lipid particle. A covalent attachment includes the sharing of electrons in a chemical bond. Non-covalent interactions include dispersed electromagnetic interactions such as hydrogen bonds, ionic bonds, van der Waals interactions, and hydrophobic bonds.

In some embodiments, an active ingredient is encapsulated in the lipid particle. The term “encapsulate” means to enclose, surround or encase. As it relates to the formulation of the compounds of the disclosure, encapsulation may be substantial, complete or partial. The term “substantially encapsulated” or “substantial encapsulation” means that greater than 50%, 60%, 70%, 80%, 85%, 90%, 95%, 96%, 97%, 98%, 99%, 99.9%, or greater of the pharmaceutical composition or active ingredient of the disclosure may be enclosed, surrounded, or encased within a delivery agent (e.g., liposome or LNP). The term “partially encapsulated” or “partial encapsulation” means that less than 50%, 40%, 30%, 20%, 10%, or less of the pharmaceutical composition or active ingredient of the disclosure may be enclosed, surrounded, or encased within the delivery agent. Encapsulation may be determined by measuring the escape or the activity of the pharmaceutical composition or active ingredient of the disclosure using fluorescence and/or electron microscopy. For example, at least 1%, 5%, 10%, 20%, 30%, 40%, 50%, 60%, 70%, 80%, 85%, 90%, 95%, 96%, 97%, 98%, 99%, 99.9%, or greater of the pharmaceutical composition or active ingredient of the disclosure is encapsulated in a delivery agent (e.g., liposome or LNP).

Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro (2011) Journal of drug delivery 2011).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro (2011) Journal of drug delivery 2011).

A conventional liposome formulation is mainly comprised of natural phospholipids and phospholipids such as 1,2-distearoryl-sn-glycero-3-phosphatidyl choline (DSPC), sphingomyelin, egg phosphatidylcholines, and monosialoganglioside. In some embodiments, 1,2-dioleoyl-sn-glycero-3-phosphoethanolamine (DOPE) increases stability of a liposome.

Additives may be added to liposomes in order to modify their structure and properties. In some embodiments, cholesterol and/or sphingomyelin may be added to a liposomal mixture to help stabilize the liposomal structure and to prevent leakage of the liposomal inner cargo. In some embodiments, addition of cholesterol to a conventional liposome formulation reduces rapid release of the encapsulated active ingredient (i.e. guide RNAs and/or RGNs, or polynucleotides comprising or encoding such) into the plasma. In some embodiments, liposomes are prepared from hydrogenated egg phosphatidylcholine or egg phosphatidylcholine, cholesterol, and dicetyl phosphate. In some embodiments, mean liposome vesicle size is adjusted to about 50 or 100 nm.

In some embodiments, Trojan Horse liposomes (also known as Molecular Trojan Horses or PEGylated immunoliposomes) may be used in pharmaceutical compositions for delivery of an active ingredient across the BBB (described on World Wide Web at cshprotocols.cshlp.org/content/2010/4/pdb.prot5407.long). Without being bound by any theory, it is believed that neutral lipid particles with specific antibodies conjugated to the surface allows crossing of the BBB via endocytosis. In some embodiments, pharmaceutical compositions comprising Trojan Horse liposomes may be used to deliver an active ingredient (i.e. guide RNAs and/or RGNs, or polynucleotides comprising or encoding such) to the brain via an intravascular injection.

In some embodiments, liposomes include stable nucleic-acid-lipid particles (SNALP) (see, e.g., Morrissey et al. (2005) Nature Biotechnology 23(8):1002-1007; Zimmerman et al. (2006) Nature 441: 111-114). SNALPs include a mixture of cationic and fusogenic lipids and coated with polyethylene glycol (PEG) that allow cellular uptake and endosomal release of an active ingredient cargo. In some embodiments, a SNALP is a class of LNP and includes an ionizable lipid that is cationic at low pH (e.g., DLinDMA), a neutral helper lipid, cholesterol, and a diffusible polyethylene glycol (PEG)-lipid. In some embodiments, a SNALP formulation includes the following lipids: 3-N-(-methoxy poly(ethylene glycol)2000) carbamoyl-1,2-dimyrestyloxy-propylamine (PEG-cDMA); 1,2-dilinoleyloxy-N,N-dimethyl-3-aminopropane (DLinDMA); 1,2-distearoyl-sn-glycero-3-phosphocholine (DSPC); and cholesterol. In some embodiments, a SNALP includes synthetic cholesterol, dipalmitoylphosphatidylcholine (DOPC), PEG-cDMA, and DLinDMA (see, e.g., Geisbert et al. (2010) Lancet 375:1896-1905). In some embodiments, a SNALP includes synthetic cholesterol, DSPC, PEG-cDMA, and DLinDMA (see, e.g., Judge et al. (2009) J. Clin. Invest. 119:661-673). In some embodiments, SNALP liposomes are about 80-100 nm in size. SNALPs have been used as effective delivery molecules to highly vascularized HepG2-derived liver tumors (see, e.g., Li et al. (2012) Gene Therapy 19:775-780).

Without being bound by any one theory, during formulation of SNALPs, the ionizable lipid serves to condense lipid with an active ingredient (e.g., a nucleic acid molecule) during particle formation. When positively charged under increasingly acidic endosomal conditions, the ionizable lipid may mediate the fusion of a SNALP with the endosomal membrane, enabling release of the active ingredient into the cytoplasm. The PEG-lipid may stabilize the particle and reduce aggregation during formulation, and subsequently may provide a neutral hydrophilic exterior that improves pharmacokinetic properties. In some embodiments, SNALP liposomes are prepared by formulating DLinDMA and PEG-cDMA with DSPC, cholesterol and an active ingredient using a 25:1 lipid: active ingredient ratio and a 48:40:10:2 molar ratio of cholesterol:DLinDMA:DSPC:PEG-cDMA.

In some embodiments, a pharmaceutical composition of the disclosure includes LNPs. In some embodiments, lipids may be formulated with an active ingredient of the present disclosure to form LNPs. An LNP comprises a plurality of lipid molecules physically associated with each other by intermolecular forces. In some embodiments, LNPs include liposomes. In some embodiments, LNPs differ from liposomes in not having a continuous lipid bilayer. In some embodiments, LNPs comprise solid particles having a mixture of solid and liquid lipids. In some embodiments, LNPs include dendrimer lipid nanoparticles (DLNPs), SNALPs, and lipid-like nanoparticles (LLNPs). In general, a “nanoparticle” refers to any particle having a diameter of less than 1000 nanometers (nm). In some embodiments, nanoparticles have a diameter of 500 nm or less. In some embodiments, nanoparticles have a diameter ranging between 25 nm and 200 nm, or 100 nm or less. In some embodiments, nanoparticles have a diameter ranging between 35 nm and 60 nm. In some embodiments, an LNP includes a lipid particle between about 1 and about 100 nm in size.

LNPs include four components: ionizable cationic lipids, fusogenic zwitterionic phospholipids, cholesterol, and PEGylated (PEG) lipids. In some embodiments, the ionizable cationic lipid component complexes a negatively charged polynucleotide and enhances endosomal escape). In some embodiments, the phospholipid component functions in modifying lipid bilayer structure. In some embodiments, the cholesterol component helps to stabilize an LNP. In some embodiments, the PEG lipid component decreases LNP aggregation and non-specific uptake.

Ionizable cationic lipids useful in LNPs include: 1,2-dilineoyl-3-dimethylammonium-propane (DLinDAP); DLinDMA; 1,2-dilinoleyloxy-keto-N,N-dimethyl-3-aminopropane (DLinK-DMA); 1,2-dilinoleyl-4-(2-dimethylaminoethyl)-[1,3]-dioxolane (DLinKC2-DMA); 5A2-SC8 (Zhou et al. (2016) Proc. Natl Acad. Sci. USA 113:520-525); C12-200 (Love et al. (2010) Proc. Natl Acad. Sci. USA 107:1864-1869); 246C10 (Kim et al. (2021) Sci Adv 7(9): eabf4398); cKK-E12 (Fenton et al. (2016) Advanced Materials 28(15):2939-2943); 1,2-distearyloxy-N,N-dimethyl-3-aminopropane (DSDMA); 1,2-dioleyloxy-N,N-dimethyl-3-aminopropane (DODMA); 1,2-dilinolenyloxy-N,N-dimethyl-3-aminopropane (DLenDMA); and dilinoleylmethyl-4-dimethylaminobutyrate (Dlin-MC3-DMA; Jayaraman et al. (2012) Angew Chem Int Ed Engl. 51(34): 8529-8533). Cationic lipids are further described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302 and 7,404,969 and US Patent Publication No. US20100036115, each of which is herein incorporated by reference in their entirety.

Zwitterionic phospholipids useful for LNPs include DSPC, DOPE, and DOPC.

PEG lipids useful for LNPs include: 1,2-dimyristoyl-rac-glycero-3-methoxypolyethylene glycol (PEG-DMG); (3-o-[2″-(methoxypolyethyleneglycol 2000) succinoyl]-1,2-dimyristoyl-sn-glycol (PEG-S-DMG); R-3-[(ω-methoxy-poly(ethylene glycol)2000) carbamoyl]-1,2-dimyristyloxlpropyl-3-amine (PEG-C-DOMG); and C16 PEG-ceramide. In some embodiments, an LNP includes 50:10:38.5:1.5 molar ratio of DLinKC2-DMA or C12-200:DSPC:cholesterol:PEG-DMG (see, e.g., Basha et al. (2011) Molecular Therapy 19(12):2186-2200). In some embodiments, an LNP includes 26.5:20:52:1.5 ionizable lipid:DOPE:cholesterol:PEG lipid (see, e.g., Han et al. (2022) Sci Adv 8(3): eabj6901; Kim et al. (2021) Sci Adv 7(9): eabf4398). PEG lipids are further described in WO2012099755. In some embodiments, the ratio of PEG in the LNP formulations may be increased or decreased and/or the carbon chain length of the PEG lipid may be modified from C14 to C18 to alter the pharmacokinetics and/or biodistribution of the LNP formulations.

In some embodiments, the charge of an LNP is taken into consideration. Cationic lipids may combine with negatively charged lipids to induce non-bilayer structures that facilitate intracellular delivery. Because charged LNPs are rapidly cleared from circulation following intravenous injection, ionizable cationic lipids with pKa values below 7 were developed (see, e.g., Basha et al. (2011) Molecular Therapy 19(12):2186-2200). Negatively charged polymers such as polynucleotides may be loaded into LNPs at low pH values (e.g., pH 4) where the ionizable lipids display a positive charge. However, at physiological pH values, the LNPs exhibit a low surface charge compatible with longer circulation times.

Preparation of LNPs and encapsulation of an active ingredient are described in e.g., Basha et al. (2011) Molecular Therapy 19(12):1286-2200; Han et al. (2022) Sci Adv 8(3): eabj6901; Kim et al. (2021) Sci Adv 7(9): eabf4398; Finn et al. (2018) Cell Reports 22:2227-2235; Wei et al. (2020) Nature Communications 11:3232; WO2011127255; and WO2008103276. Lipids are commercially available (e.g., from Tekmira Pharmaceuticals, Vancouver, Canada; Avanti Polar Lipids, Inc., Alabaster, AL) or may be synthesized (e.g., Kim et al. (2021) Sci Adv 7(9): eabf4398). Synthesis of cationic lipids are also described in International Publication Nos. WO2012040184, WO2011153120, WO2011149733, WO2011090965, WO2011043913, WO2011022460, WO2012061259, WO2012054365, WO2012044638, WO2010080724 and WO201021865. Cholesterol is commercially available (e.g., from Sigma-Aldrich, St Louis, MO).

In some embodiments, encapsulation may be performed by dissolving lipid mixtures comprising cationic lipid (e.g., Dlin-DMA):phospholipid (e.g., DSPC, DOPE):cholesterol:PEG-lipid (e.g., at 40:10:40:10 molar ratio) in ethanol. An active ingredient (e.g., a polynucleotide comprising or encoding a guide RNA or RGN of the disclosure) may be dissolved in an acidic buffer (e.g., citrate, acetate), pH 3 or 4. In some embodiments, the lipid solution and active ingredient solution may be mixed using a microfluidics system (Chen et al. (2012) J. Amer. Chem. Soc. 134:6948-6951; e.g., NanoAssemblr from Precision Nanosystems) or by dropwise addition of the lipid solution to the active ingredient solution. Removal of ethanol and neutralization of formulation buffer may be performed by dialysis for, e.g., 16 hours or overnight, against phosphate-buffered saline (PBS) using dialysis cassettes (e.g., 3500 molecular weight cut-off cassettes from Life Technologies). Dynamic light scattering may be used to assess LNP size, polydispersity index (PDI), and zeta potential. Encapsulation efficiency of an active ingredient such as RNA may be determined by assays such as Quant-It™ Ribogreen Assay (Thermo Fisher). In some embodiments wherein the encapsulated active ingredient is a polynucleotide, the polynucleotide may be extracted from the eluted nanoparticles and quantified at 260 nm. LNP pKa may be assessed using a 2-(p-toluidino)-6-napthalene sulfonic acid (TNS) assay (Zhang et al. (2011) Langmuir 27(5):1907-1914). In some embodiments, a final lipid: active ingredient weight ratio includes 12:1, 11:1, 10:1, 9:1, 8:1, 7:1, 6:1, and 5:1.

In some embodiments where a pharmaceutical composition comprises a ribonucleoprotein (RNP) complex (i.e. RGN and guide RNA) encapsulated in an LNP, inclusion of an additional permanent cationic lipid (e.g., 1,2-dioleoyl-3-trimethylammonium-propane (DOTAP)) allows formation of LNPs comprising RNP by mixing an ethanol solution of lipids with a solution of RNP at physiological pH (e.g., PBS buffer; Wei et al. (2020) Nature Communications 11:3232). In some embodiments, the permanent cationic lipid is included at 10 to 20 mole % of total lipids in LNPs.

In some embodiments, the LNP formulations described herein may additionally comprise a permeability enhancer molecule. Non-limiting permeability enhancer molecules are described in US2005/0222064.

In some embodiments, the LNP compositions are biodegradable, in that they do not accumulate to cytotoxic levels in vivo at a therapeutically effective dose. LNP formulations may be improved by replacing the cationic lipid with a biodegradable cationic lipid which is known as a rapidly eliminated lipid nanoparticle (reLNP). In some embodiments, the rapid metabolism of the rapidly eliminated lipids can improve the tolerability and therapeutic index of LNPs by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In some embodiments, the LNP compositions do not cause an innate immune response that leads to substantial adverse effects at a therapeutic dose level. In some embodiments, the LNP compositions provided herein do not cause toxicity at a therapeutic dose level.

In some embodiments, the active ingredient (i.e. guide RNAs and/or RGNs, or polynucleotides comprising or encoding such) is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see, e.g., Zhang et al. (2008) ACS Nano 2(8):1696-1702).

In some embodiments, a lipid-based formulation including an active ingredient (i.e. guide RNAs and/or RGNs, or polynucleotides comprising or encoding such) can be formulated for controlled release and/or targeted delivery. As used herein, “controlled release” refers to a pharmaceutical composition or compound release profile that conforms to a particular pattern of release to effect a therapeutic outcome.

In some embodiments, a lipid-based formulation including an active ingredient (i.e. guide RNAs and/or RGNs, or polynucleotides comprising or encoding such) includes at least one controlled release coating. Controlled release coatings include: OPADRY® (Colorcon Inc., Harleysville, PA); polyvinylpyrrolidone/vinyl acetate copolymer; polyvinylpyrrolidone; hydroxypropyl methylcellulose; hydroxypropyl cellulose; hydroxyethyl cellulose; EUDRAGIT RL® (Evonik, Essen, Germany); EUDRAGIT RS® (Evonik, Essen, Germany); and cellulose derivatives such as ethylcellulose aqueous dispersions (AQUACOAT® and SURELEASE®, Colorcon Inc., Harleysville, PA). In some embodiments, the controlled release and/or targeted delivery formulation may comprise at least one degradable polyester which may contain polycationic side chains. Degradable polyesters include poly(serine ester), poly(L-lactide-co-L-lysine), poly(4-hydroxy-L-proline ester), and combinations thereof. In some embodiments, the degradable polyesters may include a PEG conjugation to form a PEGylated polymer.

In some embodiments, LNP formulations may be prepared such that they passively or actively are directed to different cell types in vivo, including hepatocytes, immune cells, tumor cells, endothelial cells, antigen presenting cells, and leukocytes (Akinc et al. (2010) Mol Ther. 18: 1357-1364; Song et al. (2005) Nat Biotechnol. 23:709-717; Judge et al. (2009) J Clin Invest. 119:661-673; Kaufmann et al. (2010) Microvasc Res 80:286-293; Santel et al. (2006) Gene Ther 13:1222-1234; Santel et al. (2006) Gene Ther 13:1360-1370; Gutbier et al. (2010) Pulm Pharmacol. Ther. 23:334-344; Basha et al. (2011) Mol. Ther. 19:2186-2200; Fenske and Cullis (2008) Expert Opin Drug Deliv. 5:25-44; Peer et al. (2008) Science 319:627-630; Peer and Lieberman (2011) Gene Ther. 18:1127-1133; all of which are incorporated herein by reference in their entirety). One example of passive targeting of formulations to liver cells includes the DLin-DMA, DLin-KC2-DMA and MC3-based lipid nanoparticle formulations which have been shown to bind to apolipoprotein E and promote binding and uptake of these formulations into hepatocytes in vivo (Akinc et al. (2010) Mol Ther. 18: 1357-1364).

LNP formulations can also be selectively targeted through expression of different ligands on their surface such as folate, transferrin, N-acetylgalactosamine (GalNAc), and antibody targeted approaches (Kolhatkar et al. (2011) Curr Drug Discov Technol. 8:197-206; Musacchio and Torchilin (2011) Front Biosci. 16:1388-1412; Yu et al. (2010) Mol Membr Biol. 27:286-298; Patil et al. (2008) Crit Rev Ther Drug Carrier Syst. 25:1-61; Benoit et al. (2011) Biomacromolecules. 12:2708-2714; Zhao et al. (2008) Expert Opin Drug Deliv. 5:309-319; Akinc et al. (2010) Mol Ther. 18: 1357-1364; Srinivasan et al. (2012) Methods Mol Biol. 820:105-116; Ben-Arie et al. (2012) Methods Mol Biol. 757:497-507; Peer, D (2010) J of controlled release 148(1):63-68; Peer et al. (2007) Proc Natl Acad Sci USA. 104:4095-4100; Kim et al. (2011) Methods Mol Biol. 721:339-353; Subramanya et al. (2010) Mol Ther. 18:2028-2037; Song et al. (2005) Nat Biotechnol. 23:709-717; Peer et al. (2008) Science 319:627-630; Peer and Lieberman (2011) Gene Ther. 18:1127-1133; all of which are incorporated herein by reference in their entirety).

In some embodiments, an active ingredient (i.e. guide RNAs and/or RGNs, or polynucleotides comprising or encoding such) may be encapsulated into an LNP and the LNP may then be encapsulated into a polymer, polymer matrix, hydrogel and/or surgical sealant described herein and/or known in the art. In some embodiments, the polymer, hydrogel or surgical sealant includes: poly(lactic-co-glycolic acid (PLGA); ethylene vinyl acetate (EVAc); poloxamer; GELSITE® (Nanotherapeutics, Inc. Alachua, FL); HYLENEX® (Halozyme Therapeutics, San Diego CA); surgical sealants such as fibrinogen polymers (Ethicon Inc., Cornelia, GA) and TISSELL® (Baxter International, Inc Deerfield, IL); PEG-based sealants; and COSEAL® (Baxter International, Inc Deerfield, IL).

LNPs and LNP formulations are further described in, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658; European Pat. Nos. 1766035; 1519714; 1781593; and 1664316.

In some embodiments wherein cells comprising or modified with the presently disclosed expression constructs (e.g., encoding gene silencing RNAs, RGN, gRNAs, RGN systems) are administered to a subject, the cells are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells described herein using routine experimentation.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable and pharmaceutically acceptable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

The presently disclosed expression constructs can be formulated with pharmaceutically acceptable excipients such as carriers, solvents, stabilizers, adjuvants, diluents, etc., depending upon the particular mode of administration and dosage form. In some embodiments, these pharmaceutical compositions are formulated to achieve a physiologically compatible pH, and range from a pH of about 3 to a pH of about 11, about pH 3 to about pH 7, depending on the formulation and route of administration. In some embodiments, the pH can be adjusted to a range from about pH 5.0 to about pH 8. In some embodiments, the compositions can comprise a therapeutically effective amount of at least one compound as described herein, together with one or more pharmaceutically acceptable excipients. In some embodiments, the compositions comprise a combination of the compounds described herein, or include a second active ingredient useful in the treatment or prevention of bacterial growth (for example and without limitation, anti-bacterial or anti-microbial agents), or include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, the formulations are provided in unit-dose or multi-dose containers, for example sealed ampules and vials, and may be stored in a freeze-dried (lyophilized) condition requiring the addition of the sterile liquid carrier, for example, saline, water-for-injection, a semi-liquid foam, or gel, immediately prior to use. Extemporaneous injection solutions and suspensions may be prepared from sterile powders, granules and tablets of the kind previously described. In some embodiments, the active ingredient is dissolved in a buffered liquid solution that is frozen in a unit-dose or multi-dose container and later thawed for injection or kept/stabilized under refrigeration until use.

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art.

Methods of treating a disease in a subject in need thereof are provided herein. The methods comprise administering to a subject in need thereof an effective amount of a presently disclosed polynucleotide(s) comprising an expression construct comprising a presently disclosed promoter (i.e., SEQ ID NOs: 1-10 or an active variant or fragment of any thereof) operably linked to a coding sequence (e.g., coding sequence for a gene silencing RNA, a guide RNA or a crRNA, a polypeptide such as an RGN), or a cell modified by or comprising any one of these compositions.

In some embodiments, the treatment comprises in vivo gene editing by administering one or more polynucleotides comprising an expression construct comprising a presently disclosed promoter operably linked to a guide RNA or a crRNA, and in some embodiments, a second expression construct comprising an RGN-coding sequence and/or a tracrRNA-coding sequence. In some embodiments, the treatment comprises ex vivo gene editing wherein cells are genetically modified ex vivo with an RGN and then the modified cells are administered to a subject. In some embodiments, the genetically modified cells originate from the subject that is then administered the modified cells, and the transplanted cells are referred to herein as autologous. In some embodiments, the genetically modified cells originate from a different subject (i.e., donor) within the same species as the subject that is administered the modified cells (i.e., recipient), and the transplanted cells are referred to herein as allogeneic. In some examples described herein, the cells can be expanded in culture prior to administration to a subject in need thereof.

In some embodiments, the disease to be treated with the presently disclosed compositions is one that can be treated with immunotherapy, such as with a chimeric antigen receptor (CAR) T cell. Such diseases include but are not limited to cancer.

In some embodiments, the disease to be treated with the presently disclosed compositions is associated with a causal mutation. As used herein, a “causal mutation” refers to a particular nucleotide, nucleotides, or nucleotide sequence in the genome that contributes to the severity or presence of a disease or disorder in a subject. The correction of the causal mutation leads to the improvement of at least one symptom resulting from a disease or disorder. In some embodiments, the causal mutation is adjacent to a PAM site recognized by an RGN. The causal mutation can be corrected with an RGN or a fusion polypeptide comprising an RGN and a base-editing polypeptide (i.e., a base editor). Non-limiting examples of diseases associated with a causal mutation include cystic fibrosis, Hurler syndrome, Friedreich's Ataxia, Huntington's Disease, and sickle cell disease. Additional non-limiting examples of disease-associated genes and mutations are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the disease to be treated with the presently disclosed compositions, such as those comprising a coding sequence for a gene silencing RNA, is a disease associated with the overexpression of a particular gene. In some of these embodiments, the gene silencing RNA targets the overexpressed gene.

In some embodiments, a method of treating a disease in a subject in need thereof comprises creating an induced pluripotent stem cell (iPSC) or isolating a mesenchymal stem cell from the subject, contacting the iPSC or mesenchymal stem cell with an RGN polypeptide complexed or with a pharmaceutical composition disclosed herein in order to genetically modify a target sequence within the cell, differentiating the modified iPSC or the modified mesenchymal stem cell into a genetically-modified mature cell or precursor thereof, and administering the genetically-modified mature cell or precursor thereof into the subject. In some embodiments, the iPSC or the mesenchymal stem cell is an autologous or an allogeneic cell. In some embodiments, the iPSC or the mesenchymal stem cell is derived from a donor that is a perfect human leukocyte antigen (HLA) match for the subject. In some embodiments, the subject is administered a myeloablative therapy prior to administration of the modified cells.

Any method known in the art for creating patient specific iPS cells can be used, including but not limited to that described in Takahashi and Yamanaka, Cell 126(4):663-76, 2006. For example, the creating step can comprise: a) isolating a somatic cell, such as a skin cell or fibroblast, from the subject; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28. Mesenchymal stem cells can be isolated according to any method known in the art, such as from a patient's bone marrow or peripheral blood. For example, marrow aspirate can be collected into a syringe with heparin. Cells can be washed and centrifuged on a Percoll. The cells can be cultured in Dulbecco's modified Eagle's medium (DMEM) (low glucose) containing 10% fetal bovine serum (FBS) (Pittinger M F, Mackay A M, Beck S C et al., Science 1999; 284:143-147).

Genetically modified cells of the disclosure administered to a subject include autologous and allogeneic cells. Allogeneic cells refer to cells that are from a donor or donors (i.e. an individual or individuals from which the genetically modified cells are derived). Autologous cells refer to cells that are from the subject undergoing treatment (i.e. the recipient of the genetically modified cells). Due to the risk of transplant rejection, an effort is made to optimize the degree of major histocompatibility complex (MHC)/human leukocyte antigen (HLA) matching between donor tissue and recipient. HLA are found on the surface of cells and help the body in identifying self versus non-self, so that the body can attack foreign entities such as bacteria and viruses. HLA typing of donor tissue and the recipient concerns determining the genotype of six HLA antigens or alleles between a donor(s) and recipient to assess the degree to which the six HLA match. HLA alleles usually refer to two each at the loci HLA-A, HLA-B and HLA-DR, or one each at the loci HLA-A, HLA-B and HLA-C and one each at the loci HAL-DRB1, HLA-DQB1 and HLA-DPB1 (see e.g., Kawase et al., 2007, Blood 110:2235-2241). In some embodiments, 4 of 6 HLA matching between donor(s) and recipient are sufficient for administration to the recipient of cells derived from a donor. In some embodiments, 5 of 6 HLA matching between donor(s) and recipient are sufficient for administration to the recipient of cells derived from a donor(s). In some embodiments, 6 of 6 HLA are matched between donor(s) and recipient for administration to the recipient of cells derived from the donor(s). In general, a 4/6, 5/6, or a 6/6 HLA match is the standard of clinical care. When all 6 HLA match between donor(s) and recipient, the match is referred to as being a perfect match.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. In some embodiments, treatment may be administered after one or more symptoms have developed and/or after a disease has been diagnosed. In some embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The term “administering” refers to the placement of an active ingredient into a subject, by a method or route that results in at least partial localization of the introduced active ingredient at a desired site, such as a site of injury or repair, such that a desired effect(s) is produced. In some embodiments, the disclosure provides methods comprising delivering any of the nucleic acid molecules, expression constructs, RGN polypeptides, ribonucleoprotein complexes, vectors, and/or pharmaceutical compositions described herein. In some embodiments, the delivering comprises electroporation. In some embodiments, the disclosure further provides cells produced by such methods, and organisms (such as animals or plants) comprising or produced from such cells. In some embodiments, a RGN polypeptide and/or nucleic acid molecules as described herein in combination with (and optionally complexed with) a guide sequence is delivered to a cell.

In those embodiments wherein cells are administered, the cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment. For example, in some aspects described herein, an effective amount of photoreceptor cells or retinal progenitor cells is administered via a systemic route of administration, such as an intraperitoneal or intravenous route.

In some embodiments, the administering comprises administering by viral delivery. In some embodiments, the administering comprises administering by electroporation. In some embodiments, the administering comprises administering by nanoparticle delivery. In some embodiments, the administering comprises administering by liposome delivery. In some embodiments, administration of a pharmaceutical composition of the disclosure includes daily intravenous injections of about 1, 2, 3, 4, 5, 6, 7, 8, 9, 10 mg/kg/day, or more of an active ingredient in a pharmaceutical composition comprising a liposome or LNP. In some embodiments, administration of a pharmaceutical composition comprising a liposome or LNP includes doses of about 0.01 to 1 mg per kg of body weight. In some embodiments, administration of a pharmaceutical composition comprising a liposome or LNP includes doses of about 1 to 10 mg per kg of body weight.

Suitable routes of administering the pharmaceutical compositions described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In embodiments, the pharmaceutical composition described herein is administered to a subject by injection, inhalation (e.g., of an aerosol), by means of a catheter, by means of a suppository, or by means of an implant, the implant being of a porous, non-porous, or gelatinous material, including a membrane, such as a sialastic membrane, or a fiber. In embodiments, the pharmaceutical composition is formulated for delivery to a subject, e.g., for gene editing.

In embodiments, the pharmaceutical composition is formulated in accordance with routine procedures as a composition adapted for intravenous or subcutaneous administration to a subject, e.g., a human. In embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

In embodiments, the pharmaceutical composition can be contained within a lipid particle or vesicle, such as a liposome or microcrystal, which is also suitable for parenteral administration.

Although the descriptions of pharmaceutical compositions provided herein are principally directed to pharmaceutical compositions which are suitable for administration to humans, it will be understood by the skilled artisan that such compositions are generally suitable for administration to animals or organisms of all sorts.

As used herein, the term “subject” refers to any individual for whom diagnosis, treatment or therapy is desired. In some embodiments, the subject is an animal. In some embodiments, the subject is a mammal. In some embodiments, the subject is a human being.

The efficacy of a treatment can be determined by the skilled clinician. However, a treatment is considered an “effective treatment,” if any one or all of the signs or symptoms of a disease or disorder are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art. Treatment includes: (1) inhibiting the disease, e.g., arresting, or slowing the progression of symptoms; or (2) relieving the disease, e.g., causing regression of symptoms; and (3) preventing or reducing the likelihood of the development of symptoms.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:
1. A promoter comprising a nucleotide sequence having at least 90% sequence identity to the nucleotide sequence set forth as any one of SEQ ID NOs: 6-10.
2. The promoter of embodiment 1, wherein said promoter comprises a nucleotide sequence having at least 95% sequence identity to the nucleotide sequence set forth as any one of SEQ ID NOs: 6-10.
3. The promoter of embodiment 1, wherein said promoter comprises a nucleotide sequence having 100% sequence identity to the nucleotide sequence set forth as any one of SEQ ID NOs: 6-10.
4. The promoter of any one of embodiments 1-3, wherein said promoter comprises a TATA box sequence.
5. The promoter of any one of embodiments 1-4, wherein said promoter comprises an octamer (OCT) sequence.
6. The promoter of any one of embodiments 1-5, wherein said promoter comprises a SPH sequence.
7. The promoter of any one of embodiments 1-6, wherein said promoter comprises a proximal sequence element (PSE).
8. A vector comprising the promoter of any one of embodiments 1-7.
9. The vector of embodiment 8, wherein said vector is a viral vector.
10. The vector of embodiment 9, wherein said viral vector is an adeno-associated viral (AAV) vector.
11. An expression construct comprising the promoter of any one of embodiments 1-7 and a coding sequence, wherein said coding sequence is operably linked to said promoter.
12. The expression construct of embodiment 11, wherein said coding sequence is heterologous to said promoter.
13. The expression construct of embodiment 11 or 12, wherein said coding sequence encodes an RNA.
14. The expression construct of embodiment 13, wherein said RNA comprises a guide RNA.
15. The expression construct of embodiment 11 or 12, wherein said coding sequence encodes a polypeptide.
16. A polynucleotide comprising the expression construct of embodiment 14 and an RNA-guided nuclease (RGN)-encoding nucleotide sequence, wherein said RGN is capable of binding said guide RNA.
17. The polynucleotide of embodiment 16, wherein said polynucleotide further comprises an RNA polymerase II (pol II) promoter operably linked to the RGN-encoding nucleotide sequence.
18. The polynucleotide of embodiment 16 or 17, wherein said RGN is fused to a base-editing polypeptide.
19. The polynucleotide of embodiment 18, wherein said base-editing polypeptide comprises a cytosine deaminase or adenine deaminase.
20. The polynucleotide of embodiment 16 or 17, wherein said RGN is fused to a transcriptional activator or transcriptional repressor.
21. The polynucleotide of embodiment 16 or 17, wherein said RGN is fused to a detectable label.
22. The expression construct of embodiment 13, wherein said RNA is a crRNA.
23. A polynucleotide comprising the expression construct of embodiment 22 and a tracrRNA-encoding nucleotide sequence.
24. The polynucleotide of embodiment 23, wherein said polynucleotide further comprises an RNA polymerase III (pol III) promoter operably linked to said tracrRNA-encoding nucleotide sequence.
25. The polynucleotide of embodiment 24, wherein said pol III promoter comprises a nucleotide sequence having at least 90% sequence identity to the nucleotide sequence set forth as any one of SEQ ID NOs: 1-10.
26. The polynucleotide of embodiment 24, wherein said pol III promoter comprises a nucleotide sequence having at least 95% sequence identity to the nucleotide sequence set forth as any one of SEQ ID NOs: 1-10.
27. The polynucleotide of embodiment 24, wherein said pol III promoter comprises a nucleotide sequence having 100% sequence identity to the nucleotide sequence set forth as any one of SEQ ID NOs: 1-10.
28. The polynucleotide of any one of embodiments 23-27, wherein said polynucleotide further comprises an RNA-guided nuclease (RGN)-encoding nucleotide sequence, wherein said RGN is capable of binding said crRNA and said tracrRNA.
29. The polynucleotide of embodiment 28, wherein said polynucleotide further comprises an RNA polymerase II (pol II) promoter operably linked to said RGN-encoding nucleotide sequence.
30. The polynucleotide of embodiment 28 or 29, wherein said RGN is fused to a base-editing polypeptide.
31. The polynucleotide of embodiment 30, wherein said base-editing polypeptide comprises a cytosine deaminase or adenine deaminase.
32. The polynucleotide of embodiment 28 or 29, wherein said RGN is fused to a transcriptional activator or transcriptional repressor.
33. The polynucleotide of embodiment 28 or 29, wherein said RGN is fused to a detectable label.
34. The expression construct of embodiment 13, wherein said RNA is a tracrRNA.
35. A polynucleotide comprising the expression construct of embodiment 34 and a crRNA-encoding nucleotide sequence.
36. The polynucleotide of embodiment 35, wherein said polynucleotide further comprises an RNA polymerase III (pol III) promoter operably linked to said crRNA-encoding nucleotide sequence.
37. The polynucleotide of embodiment 36, wherein said pol III promoter comprises a nucleotide sequence having at least 90% sequence identity to the nucleotide sequence set forth as any one of SEQ ID NOs: 1-10.
38. The polynucleotide of embodiment 36, wherein said pol III promoter comprises a nucleotide sequence having at least 95% sequence identity to the nucleotide sequence set forth as any one of SEQ ID NOs: 1-10.
39. The polynucleotide of embodiment 36, wherein said pol III promoter comprises a nucleotide sequence having 100% sequence identity to the nucleotide sequence set forth as any one of SEQ ID NOs: 1-10.
40. The polynucleotide of any one of embodiments 35-39, wherein said polynucleotide further comprises an RNA-guided nuclease (RGN)-encoding nucleotide sequence, wherein said RGN is capable of binding to said crRNA and said tracrRNA.
41. The polynucleotide of embodiment 40, wherein said polynucleotide further comprises an RNA polymerase II (pol II) promoter operably linked to said RGN-encoding nucleotide sequence.
42. The polynucleotide of embodiment 40 or 41, wherein said RGN is fused to a base-editing polypeptide.
43. The polynucleotide of embodiment 42, wherein said base-editing polypeptide comprises a cytosine deaminase or adenine deaminase.
44. The polynucleotide of embodiment 40 or 41, wherein said RGN is fused to a transcriptional activator or transcriptional repressor.
45. The polynucleotide of embodiment 40 or 41, wherein said RGN is fused to a detectable label.
46. The expression construct of embodiment 13, wherein said RNA comprises a gene silencing RNA.
47. A vector comprising the expression construct of any one of embodiments 11-14, 22, 34, and 46 or the polynucleotide of any one of embodiments 16-21, 23-33, and 35-45.
48. The vector of embodiment 47, wherein said vector is a viral vector.
49. The vector of embodiment 48, wherein said viral vector is an adeno-associated viral (AAV) vector.
50. An expression construct comprising:
a) a promoter comprising a nucleotide sequence having at least 90% sequence identity to the nucleotide sequence set forth as any one of SEQ ID NOs: 1-5; and
b) a heterologous coding sequence operably linked to said promoter.
51. The expression construct of embodiment 50 wherein said promoter comprises a nucleotide sequence having at least 95% sequence identity to the nucleotide sequence set forth as any one of SEQ ID NOs: 1-5.
52. The expression construct of embodiment 50, wherein said promoter comprises a nucleotide sequence having 100% sequence identity to the nucleotide sequence set forth as any one of SEQ ID NOs: 1-5.
53. The expression construct of any one of embodiments 50-52, wherein said promoter comprises a TATA box sequence.
54. The expression construct of any one of embodiments 50-53, wherein said promoter comprises an octamer (OCT) sequence.
55. The expression construct of any one of embodiments 50-54, wherein said promoter comprises a SPH sequence.
56. The expression construct of any one of embodiments 50-55, wherein said promoter comprises a proximal sequence element (PSE).
57. The expression construct of any one of embodiments 50-56, wherein said heterologous coding sequence encodes an RNA.
58. The expression construct of embodiment 57, wherein said RNA comprises a guide RNA.
59. The expression construct of any one of embodiments 50-56, wherein said heterologous coding sequence encodes a polypeptide.
60. A polynucleotide comprising the expression construct of embodiment 58 and an RNA-guided nuclease (RGN)-encoding nucleotide sequence, wherein said RGN is capable of binding said guide RNA.
61. The polynucleotide of embodiment 60, wherein said polynucleotide further comprises an RNA polymerase II (pol II) promoter operably linked to the RGN-encoding nucleotide sequence.
62. The polynucleotide of embodiment 60 or 61, wherein said RGN is fused to a base-editing polypeptide.
63. The polynucleotide of embodiment 62, wherein said base-editing polypeptide comprises a cytosine deaminase or adenine deaminase.
64. The polynucleotide of embodiment 60 or 61, wherein said RGN is fused to a transcriptional activator or transcriptional repressor.
65. The polynucleotide of embodiment 60 or 61, wherein said RGN is fused to a detectable label.
66. The expression construct of embodiment 57, wherein said RNA is a crRNA.
67. A polynucleotide comprising the expression construct of embodiment 66 and a tracrRNA-encoding nucleotide sequence.
68. The polynucleotide of embodiment 67, wherein said polynucleotide further comprises an RNA polymerase III (pol III) promoter operably linked to said tracrRNA-encoding nucleotide sequence.
69. The polynucleotide of embodiment 68, wherein said pol III promoter comprises a nucleotide sequence having at least 90% sequence identity to the nucleotide sequence set forth as any one of SEQ ID NOs: 1-10.
70. The polynucleotide of embodiment 68, wherein said pol III promoter comprises a nucleotide sequence having at least 95% sequence identity to the nucleotide sequence set forth as any one of SEQ ID NOs: 1-10.
71. The polynucleotide of embodiment 68, wherein said pol III promoter comprises a nucleotide sequence having 100% sequence identity to the nucleotide sequence set forth as any one of SEQ ID NOs: 1-10.
72. The polynucleotide of any one of embodiments 67-71, wherein said polynucleotide further comprises an RNA-guided nuclease (RGN)-encoding nucleotide sequence, wherein said RGN is capable of binding to said crRNA and said tracrRNA.
73. The polynucleotide of embodiment 72, wherein said polynucleotide further comprises an RNA polymerase II (pol II) promoter operably linked to said RGN-encoding nucleotide sequence.
74. The polynucleotide of embodiment 72 or 73, wherein said RGN is fused to a base-editing polypeptide.
75. The polynucleotide of embodiment 74, wherein said base-editing polypeptide comprises a cytosine deaminase or adenine deaminase.
76. The polynucleotide of embodiment 72 or 73, wherein said RGN is fused to a transcriptional activator or transcriptional repressor.
77. The polynucleotide of embodiment 72 or 73, wherein said RGN is fused to a detectable label.
78. The expression construct of embodiment 57, wherein said RNA is a tracrRNA.
79. A polynucleotide comprising the expression construct of embodiment 78 and a crRNA-encoding nucleotide sequence.
80. The polynucleotide of embodiment 79, wherein said polynucleotide further comprises an RNA polymerase III (pol III) promoter operably linked to said crRNA-encoding nucleotide sequence.
81. The polynucleotide of embodiment 80, wherein said pol III promoter comprises a nucleotide sequence having at least 90% sequence identity to the nucleotide sequence set forth as any one of SEQ ID NOs: 1-10.
82. The polynucleotide of embodiment 80, wherein said pol III promoter comprises a nucleotide sequence having at least 95% sequence identity to the nucleotide sequence set forth as any one of SEQ ID NOs: 1-10.
83. The polynucleotide of embodiment 80, wherein said pol III promoter comprises a nucleotide sequence having 100% sequence identity to the nucleotide sequence set forth as any one of SEQ ID NOs: 1-10.
84. The polynucleotide of any one of embodiments 79-83, wherein said polynucleotide further comprises an RNA-guided nuclease (RGN)-encoding nucleotide sequence, wherein said RGN is capable of binding to said crRNA and said tracrRNA.
85. The polynucleotide of embodiment 84, wherein said polynucleotide further comprises an RNA polymerase II (pol II) promoter operably linked to said RGN-encoding nucleotide sequence.
86. The polynucleotide of embodiment 84 or 85, wherein said RGN is fused to a base-editing polypeptide.
87. The polynucleotide of embodiment 86, wherein said base-editing polypeptide comprises a cytosine deaminase or adenine deaminase.
88. The polynucleotide of embodiment 84 or 85, wherein said RGN is fused to a transcriptional activator or transcriptional repressor.
89. The polynucleotide of embodiment 84 or 85, wherein said RGN is fused to a detectable label.
90. The expression construct of embodiment 57, wherein said RNA comprises a gene silencing RNA.
91. A vector comprising the expression construct of any one of embodiments 50-59, 66, 78, and 90 or the polynucleotide of any one of embodiments 60-65, 67-77, and 79-89.
92. The vector of embodiment 91, wherein said vector is a viral vector.
93. The vector of embodiment 92, wherein said viral vector is an adeno-associated viral (AAV) vector.
94. A cell comprising the promoter of any one of embodiments 1-7, the expression construct of any one of embodiments 11-15, 22, 34, 46, 50-59, 66, 78, and 90, the polynucleotide of any one of embodiments 16-21, 23-33, 35-45, 60-65, 67-77, and 79-89, or the vector of any one of embodiments 8-10, 47-49, and 91-93.
95. The cell of embodiment 94, wherein said cell is a prokaryotic cell.
96. The cell of embodiment 94, wherein said cell is a eukaryotic cell.
97. The cell of embodiment 95, wherein said eukaryotic cell is a mammalian cell.
98. The cell of embodiment 97, wherein said mammalian cell is a human cell.
99. The cell of embodiment 98, wherein said human cell is an immune cell.
100. The cell of embodiment 99, wherein said immune cell is a stem cell.
101. The cell of embodiment 100, wherein said stem cell is an induced pluripotent stem cell.
102. The cell of embodiment 96, wherein said eukaryotic cell is an insect or avian cell.
103. The cell of embodiment 96, wherein said eukaryotic cell is a fungal cell.
104. The cell of embodiment 96, wherein said eukaryotic cell is a plant cell.
105. A plant comprising the promoter of any one of embodiments 1-7, the expression construct of any one of embodiments 11-15, 22, 34, 46, 50-59, 66, 78, and 90, the polynucleotide of any one of embodiments 16-21, 23-33, 35-45, 60-65, 67-77, and 79-89, the vector of any one of embodiments 8-10, 47-49, and 91-93, or the cell of embodiment 104.
106. A seed comprising the cell of embodiment 104.
107. A pharmaceutical composition comprising a pharmaceutically acceptable carrier and the expression construct of any one of embodiments 11-15, 22, 34, 46, 50-59, 66, 78, and 90, the polynucleotide of any one of embodiments 16-21, 23-33, 35-45, 60-65, 67-77, and 79-89, the vector of any one of embodiments 47-49 and 91-93, or the cell of any one of embodiments 97-101.
108. The pharmaceutical composition of embodiment 107, wherein the pharmaceutical composition is lipid-based.
109. The pharmaceutical composition of embodiment 108, wherein the lipid-based pharmaceutical composition comprises liposomes or lipid nanoparticles (LNPs).
110. The pharmaceutical composition of embodiment 109, wherein the expression construct, the polynucleotide, the vector, or the cell is encapsulated in, and/or non-covalently or covalently attached to the liposomes or the LNPs.
111. A method for preparing an expression construct wherein said method comprises inserting a coding sequence into a nucleic acid molecule comprising the promoter of any one of embodiments 1-7 such that the coding sequence is operably linked to said promoter.
112. A method for preparing an expression construct wherein said method comprises inserting a promoter comprising a nucleotide sequence having at least 90% sequence identity to the nucleotide sequence set forth as any one of SEQ ID NOs: 6-10 into a nucleic acid molecule comprising a coding sequence such that the coding sequence is operably linked to said promoter.
113. The method of embodiment 112, wherein said promoter comprises a nucleotide sequence having at least 95% sequence identity to the nucleotide sequence set forth as any one of SEQ ID NOs: 6-10.
114. The method of embodiment 112, wherein said promoter comprises a nucleotide sequence having 100% sequence identity to the nucleotide sequence set forth as any one of SEQ ID NOs: 6-10.
115. The method of any one of embodiments 112-114, wherein said promoter comprises a TATA box sequence.
116. The method of any one of embodiments 112-115, wherein said promoter comprises an octamer (OCT) sequence.
117. The method of any one of embodiments 112-116, wherein said promoter comprises a SPH sequence.
118. The method of any one of embodiments 112-117, wherein said promoter comprises a proximal sequence element (PSE).
119. The method of any one of embodiments 112-118, wherein said coding sequence is heterologous to said promoter.
120. The method of any one of embodiments 112-119, wherein said coding sequence encodes an RNA.
121. The method of embodiment 120, wherein said RNA comprises a guide RNA.
122. The method of embodiment 120, wherein said RNA is a crRNA.
123. The method of embodiment 120, wherein said RNA is a tracrRNA.
124. The method of embodiment 120, wherein said RNA comprises a gene silencing RNA.
125. The method of any one of embodiments 111-119, wherein said coding sequence encodes a polypeptide.
126. A method for expressing an RNA, said method comprising contacting in vitro the expression construct of any one of embodiments 13, 14, 22, 34, 46, 57, 58, 66, 78, and 90, or the polynucleotide of any one of embodiments 16-21, 23-33, 35-45, 60-65, 67-77, and 79-89 with RNA polymerase III and ribonucleotide triphosphates.
127. A method for expressing an RNA in a cell, said method comprising introducing into said cell the expression construct of any one of embodiments 13, 14, 22, 34, 46, 57, 58, 66, 78, and 90, or the polynucleotide of any one of embodiments 16-21, 23-33, 35-45, 60-65, 67-77, and 79-89.
128. A method for expressing a polypeptide in a cell, said method comprising introducing into said cell the expression construct of embodiment 15.
129. A method for making an RGN ribonucleoprotein complex in a cell, said method comprising introducing into a cell the polynucleotide of any one of embodiments 16-21, 28-33, 40-45, 60-65, 72-77, and 84-89, wherein the RGN and the gRNA form an RGN ribonucleoprotein complex.
130. The method of embodiment 129, further comprising purifying said RGN ribonucleoprotein complex.
131. A method for making an mRNA in vitro, said method comprising contacting the expression construct of embodiment 15 or 59 with RNA polymerase II and ribonucleotide triphosphates.
132. The method of embodiment 131, wherein said method further comprises contacting the expression construct with a cap analog to generate a capped mRNA.
133. The method of embodiment 131 or 132, wherein said method further comprises contacting the capped mRNA with a poly(A) polymerase to generate a capped and tailed mRNA.
134. The method of embodiment 132 or 133, wherein said method further comprises contacting said capped mRNA or said capped and tailed mRNA in vitro with a cell-free lysate to generate an in vitro translated polypeptide.
135. A method for binding a target nucleic acid molecule in a cell, wherein said target nucleic acid molecule comprises a target sequence, said method comprising introducing into said cell the polynucleotide of any one of embodiments 16-21, 28-33, 40-45, 60-65, 72-77, and 84-89, wherein said guide RNA of any one of embodiments 16-21, and 60-65 or the crRNA and tracrRNA of the polynucleotide of any one of embodiments 28-33, 40-45, 72-77, and 84-89 binds to said RGN, thereby directing said RGN to bind to said target sequence within said target nucleic acid molecule.
136. A method for cleaving and/or modifying a target nucleic acid molecule in a cell, wherein said target nucleic acid molecule comprises a target sequence, said method comprising introducing into said cell the polynucleotide of any one of embodiments 16-19, 28-31, 40-43, 60-63, 72-75, and 84-87, wherein said guide RNA of any one of embodiments 16-19, 60-63 or the crRNA and tracrRNA of the polynucleotide of any one of embodiments 28-31, 40-43, 72-75, and 84-87 binds to said RGN, thereby directing said RGN to bind to said target sequence and cleave and/or modify said target nucleic acid molecule.
137. A method for modifying the expression of a target gene in a cell, wherein said target gene comprises a target sequence, said method comprising introducing into said cell the polynucleotide of any one of embodiments 20, 32, 44, 64, 76, and 88, wherein said guide RNA of embodiment 20 or 64 or the crRNA and tracrRNA of the polynucleotide of any one of embodiments 32, 44, 76, and 88 binds to said RGN, thereby directing said RGN to said target sequence and modifying the expression of said target gene.
138. A method for detecting a target sequence in a cell, said method comprising introducing into said cell the polynucleotide of any one of embodiments 21, 33, 45, 65, 77, and 89, wherein said guide RNA of embodiment 21 or 65 or the crRNA and tracrRNA of the polynucleotide of any one of embodiments 33, 45, 77, and 89 binds to said RGN, thereby directing said RGN to said target sequence; and detecting said detectable label.
139. A method for binding a target nucleic acid molecule in a cell, wherein said target nucleic acid molecule comprises a target sequence, wherein said method comprises introducing into said cell the expression construct of embodiment 14 or 58, or the polynucleotide of any one of embodiments 23-27, 35-39, 67-71, and 79-83, wherein said cell comprises an RNA-guided nuclease (RGN); and wherein said guide RNA of embodiment 14 or 58, or the crRNA and tracrRNA of the polynucleotide of any one of embodiments 23-27, 35-39, 67-71, and 79-83 binds to said RGN, thereby directing said RGN to bind said target sequence within said target nucleic acid molecule.
140. A method for cleaving and/or modifying a target nucleic acid molecule in a cell, wherein said target nucleic acid molecule comprises a target sequence, wherein said method comprises introducing into said cell the expression construct of embodiment 14 or 58, or the polynucleotide of any one of embodiments 23-27, 35-39, 67-71, and 79-83, wherein said cell comprises:
a) an RNA-guided nuclease (RGN); or
b) a fusion protein comprising a nuclease-inactive or nickase RGN fused to a base-editing polypeptide;
and wherein said guide RNA of embodiment 14 or 58, or the crRNA and tracrRNA of the polynucleotide of any one of embodiments 23-27, 35-39, 67-71, and 79-83 binds to said RGN, thereby directing said RGN or said fusion protein to bind to said target sequence and cleave and/or modify said target nucleic acid molecule.
141. The method of embodiment 140, further comprising introducing said RGN or said fusion protein or a nucleic acid molecule encoding the same into said cell prior to or simultaneously with the introduction of said expression construct of embodiment 14 or 58, or the polynucleotide of any one of embodiments 23-27, 35-39, 67-71, and 79-83.
142. A method for modifying the expression of a target gene, wherein said target gene comprises a target sequence, wherein said method comprises introducing into said cell the expression construct of embodiment 14 or 58, or the polynucleotide of any one of embodiments 23-27, 35-39, 67-71, and 79-83, wherein said cell comprises a fusion protein comprising an RNA-guided nuclease (RGN) fused to a transcriptional activator or transcriptional repressor, and wherein said guide RNA of embodiment 14 or 58, or the crRNA and tracrRNA of the polynucleotide of any one of embodiments 23-27, 35-39, 67-71, and 79-83 binds to said RGN, thereby directing said fusion protein to said target sequence and modifying the expression of said target gene.
143. The method of embodiment 142, further comprising introducing said fusion protein or a nucleic acid molecule encoding the same into said cell prior to or simultaneously with the introduction of said expression construct of embodiment 14 or 58, or the polynucleotide of any one of embodiments 23-27, 35-39, 67-71, and 79-83.
144. A method for detecting a target sequence in a cell, wherein said method comprises introducing into said cell the expression construct of embodiment 14 or 58, or the polynucleotide of any one of embodiments 23-27, 35-39, 67-71, and 79-83, wherein said cell comprises a fusion protein comprising an RNA-guided nuclease (RGN) fused to a detectable label, and wherein said guide RNA of embodiment 14 or 58, or the crRNA and tracrRNA of the polynucleotide of any one of embodiments 23-27, 35-39, 67-71, and 79-83 binds to said RGN, thereby directing said fusion protein to said target sequence; and detecting said detectable label.
145. The method of embodiment 144, further comprising introducing said fusion protein or a nucleic acid molecule encoding the same into said cell prior to or simultaneously with the introduction of said expression construct of embodiment 14 or 58, or the polynucleotide of any one of embodiments 23-27, 35-39, 67-71, and 79-83.
146. A method for binding a target nucleic acid molecule in a cell, wherein said target nucleic acid molecule comprises a target sequence, wherein said method comprises:
a) introducing into said cell the expression construct of embodiment 14 or 58, or the polynucleotide of any one of embodiments 23-27, 35-39, 67-71, and 79-83; and
b) introducing into said cell:
   i) an RNA-guided nuclease (RGN); or
   ii) a nucleic acid molecule encoding an RGN;
   wherein said step a) occurs before, during, or after step b); and
wherein said guide RNA of embodiment 14 or 58, or the crRNA and tracrRNA of the polynucleotide of any one of embodiments 23-27, 35-39, 67-71, and 79-83 binds to said RGN, thereby directing said RGN to bind said target sequence within said target nucleic acid molecule.
147. A method for cleaving and/or modifying a target nucleic acid molecule in a cell, wherein said target nucleic acid molecule comprises a target sequence, wherein said method comprises:
a) introducing into said cell the expression construct of embodiment 14 or 58, or the polynucleotide of any one of embodiments 23-27, 35-39, 67-71, and 79-83; and
b) introducing into said cell:
   i) an RNA-guided nuclease (RGN);
   ii) a fusion protein comprising a nuclease-inactive or nickase RGN fused to a base-editing polypeptide;
   iii) a nucleic acid molecule encoding an RGN; or
   iv) a nucleic acid molecule encoding a fusion protein comprising a nuclease-inactive or nickase RGN fused to a base-editing polypeptide;
   wherein said step a) occurs before, during, or after step b); and
wherein said guide RNA of embodiment 14 or 58, or the crRNA and tracrRNA of the polynucleotide of any one of embodiments 23-27, 35-39, 67-71, and 79-83 binds to said RGN, thereby directing said RGN or said fusion protein to bind to said target sequence and cleave and/or modify said target nucleic acid molecule.
148. The method of embodiment 147, wherein said step b) iii) comprises introducing a polynucleotide comprising said nucleic acid molecule encoding said RGN or the step b) iv) comprises introducing a polynucleotide comprising said nucleic acid molecule encoding said fusion protein, and wherein said polynucleotide further comprises an RNA polymerase II (pol II) promoter operably linked to said nucleic acid molecule encoding said RGN or said nucleic acid molecule encoding said fusion protein.
149. A method for modifying the expression of a target gene in a cell, wherein said target gene comprises a target sequence, wherein said method comprises:
a) introducing into said cell the expression construct of embodiment 14 or 58, or the polynucleotide of any one of embodiments 23-27, 35-39, 67-71, and 79-83; and
b) introducing into said cell:
   i) a fusion protein comprising a nuclease-inactive RNA-guided nuclease (RGN) fused to a transcriptional activator or transcriptional repressor; or
   ii) a nucleic acid molecule encoding a fusion protein comprising a nuclease-inactive RGN fused to a transcriptional activator or transcriptional repressor;
   wherein said step a) occurs before, during, or after step b); and
   wherein said guide RNA of embodiment 14 or 58, or the crRNA and tracrRNA of the polynucleotide of any one of embodiments 23-27, 35-39, 67-71, and 79-83 binds to said RGN, thereby directing said fusion protein to said target sequence and modifying the expression of said target gene.
150. The method of embodiment 149, wherein said step b) ii) comprises introducing a polynucleotide comprising said nucleic acid molecule encoding said fusion protein, and wherein said polynucleotide further comprises an RNA polymerase II (pol II) promoter operably linked to said nucleic acid molecule encoding said fusion protein.
151. A method for detecting a target sequence in a cell, wherein said method comprises:
a) introducing into said cell the expression construct of embodiment 14 or 58, or the polynucleotide of any one of embodiments 23-27, 35-39, 67-71, and 79-83;
b) introducing into said cell:
   i) a fusion protein comprising nuclease-inactive RNA-guided nuclease (RGN) fused to a detectable label; or
   ii) a nucleic acid molecule encoding a fusion protein comprising a nuclease-inactive RGN fused to a detectable label; and
c) detecting said detectable label;
wherein step a) occurs before, during, or after step b); and
wherein said guide RNA of embodiment 14 or 58, or the crRNA and tracrRNA of the polynucleotide of any one of embodiments 23-27, 35-39, 67-71, and 79-83 binds to said RGN and directs said fusion protein to said target sequence.
152. The method of embodiment 151, wherein said step b) ii) comprises introducing into said cell a polynucleotide comprising said nucleic acid molecule encoding said fusion protein, and wherein said polynucleotide further comprises an RNA polymerase II (pol II) promoter operably linked to said nucleic acid molecule encoding said fusion protein.
153. A method for reducing the expression of a target gene in a cell, said method comprising introducing the expression construct of embodiment 46 or 90, wherein said gene silencing RNA reduces the expression of said target gene.
154. A method for treating a disease, said method comprising administering to a subject in need thereof a pharmaceutical composition comprising a pharmaceutically acceptable carrier and a polynucleotide of any one of embodiments 16-19, 28-31, 40-43, 60-63, 72-75, and 84-87.
155. The method of embodiment 154, wherein said disease is associated with a causal mutation and said pharmaceutical composition corrects said causal mutation.
156. Use of the polynucleotide of any one of embodiments 16-19, 28-31, 40-43, 60-63, 72-75, and 84-87 for the treatment of a disease in a subject.
157. The use of embodiment 156, wherein said disease is associated with a causal mutation and said treatment comprises correcting said causal mutation.
158. Use of the polynucleotide of any one of embodiments 16-19, 28-31, 40-43, 60-63, 72-75, and 84-87 for the manufacture of a medicament useful for treating a disease.
159. The use of embodiment 158, wherein said disease is associated with a causal mutation and said medicament corrects said causal mutation.
160. A method for treating a disease, said method comprising administering to a subject in need thereof a pharmaceutical composition comprising a pharmaceutically acceptable carrier and a polynucleotide of any one of embodiments 20, 32, 44, 64, 76, and 88, wherein said disease is associated with the expression of a target gene, wherein said target gene comprises a target sequence, and wherein said guide RNA of said polynucleotide of embodiment 20 or 64 or the crRNA and tracrRNA of said polynucleotide of any one of embodiments 32, 44, 76, and 88 binds to said RGN, thereby directing said RGN to said target sequence and modifying the expression of said target gene.
161. Use of the polynucleotide of any one of embodiments 20, 32, 44, 64, 76, and 88 for the treatment of a disease associated with the expression of a target gene in a subject.
162. Use of the polynucleotide of any one of embodiments 20, 32, 44, 64, 76, and 88 for the manufacture of a medicament useful for treating a disease associated with the expression of a target gene.
163. A method for treating a disease, said method comprising administering to a subject in need thereof a pharmaceutical composition comprising a pharmaceutically acceptable carrier and an expression construct of embodiment 46 or 90, wherein said disease is associated with the overexpression of a target gene, and wherein said gene silencing RNA reduces the expression of said target gene.
164. Use of the expression construct of embodiment 46 or 90 for the treatment of a disease associated with the overexpression of a target gene in a subject.
165. Use of the expression construct of embodiment 46 or 90 for the manufacture of a medicament useful for treating a disease associated with the overexpression of a target gene.

The following examples are offered by way of illustration and not by way of limitation.

### EXAMPLES

### Example 1. Demonstration of Programmable DNA Cleavage Including Editing in Eukaryotic Cells Using Novel Pol III Promoters

Novel RNA polymerase III promoters were identified and are set forth in Table 2 as SEQ ID NOs: 1-5. Truncated versions of the Pol III promoters set forth as SEQ ID NOs: 1-5 were generated and are set forth as SEQ ID NOs: 6-10, respectively.

TABLE 2
Promoter sequences

| Promoter | SEQ ID |
|---|---|
| NC_041760 | 1 |
| NW_004848155 | 2 |
| LR738627 | 3 |
| NC_044556 | 4 |
| XM_030845548 | 5 |
| Mini NC_041760 | 6 |
| Mini NW_004848155 | 7 |
| Mini LR738627 | 8 |
| Mini NC_044556 | 9 |
| Mini XM_030845548 | 10 |

Guide RNA expression constructs encoding a single gRNA from Table 3, each under the control of a polymerase III promoter from Table 2 or the human U6 promoter set forth as SEQ ID NO: 11 were synthesized and cloned into the pTwist High Copy Amp vector.

TABLE 3
Guide RNA sequences

| | | | Forward | Reverse |
|---|---|---|---|---|
| | sgRNA | Target | primer for | primer for |
| | sequence | sequence | amplification | amplification |
| | (SEQ | (SEQ | (SEQ | (SEQ |
| sgRNA ID | ID NO) | ID NO) | ID NO) | ID NO) |
| SGN000169 | 12 | 13 | 14 | 15 |
| SGN000173 | 16 | 17 | 18 | 19 |
| SGN000929 | 20 | 21 | 22 | 23 |
| SGN001101 | 24 | 25 | 26 | 27 |
| SGN000143 | 28 | 29 | 30 | 31 |
| SGN000930 | 32 | 33 | 34 | 35 |

500 ng of plasmid comprising an expression cassette encoding for an sgRNA shown in Table 4 were transfected into HEK293FT cells having stably incorporated an expression construct expressing the APG08290.1 RGN (set forth as SEQ ID NO: 52, which was disclosed in International Patent Publ. No WO 2019/236566, which is herein incorporated by reference in its entirety), with cells at 75-90% confluency in 24-well plates using Lipofectamine 2000 reagent (Life Technologies).

TABLE 4
Full Length Promoter guide RNA sequences

| | U6 sgRNA | NC_041760 | NW_004848155 | LR738627 | NC_044556 | XM_030845548 |
|---|---|---|---|---|---|---|
| sgRNA ID | ID | sgRNA ID | sgRNA ID | sgRNA ID | sgRNA ID | sgRNA ID |
| SGN000143 | SGN000143 | SGN001022 | SGN001029 | SGN001018 | SGN001025 | SGN001037 |

Cells were then incubated at 37° C. for 72 h. Following incubation, genomic DNA was extracted using NucleoSpin 96 Tissue (Macherey-Nagel) following the manufacturer's protocol. The genomic region flanking the targeted genomic site was PCR amplified using the primers in Table 3 and products were purified using ZR-96 DNA Clean and Concentrator (Zymo Research) following the manufacturer's protocol. The purified PCR products were then sent for Next Generation Sequencing on Illumina MiSeq (2×250). Results were analyzed for INDEL formation and are provided in Table 5.

TABLE 5
Programmable DNA editing with Novel Polymerase III promoters

| Promoter sgRNA | % INDEL formation |
|---|---|
| SGN001022 | 12.10% |
| SGN001029 | 9.40% |
| SGN001018 | 6.30% |
| SGN001025 | 14.20% |
| SGN001037 | 14.30% |
| SGN000143 | 13.70% |

Table 5 provides INDEL formation caused by the same sgRNA sequence, the expression of which was driven by different polymerase III promoters, demonstrating successful editing by the RGN when the sgRNA is expressed by each of these promoters.

To determine if the truncated mini promoters are active, 500 ng of plasmid comprising an expression cassette comprising a coding sequence for the APG08290.1 RGN (set forth as SEQ ID NO: 53, which was disclosed in International Patent Publ. No. WO 2019/236566) and 500 ng of plasmid comprising an expression cassette encoding for an sgRNA shown in Table 6 were co-transfected into HEK293FT cells at 75-90% confluency in 24-well plates using Lipofectamine 2000 reagent (Life Technologies).

TABLE 6
Mini Promoter guide RNA sequences

| | U6 | LR738627 | Mini |
|---|---|---|---|
| sgRNA ID | sgRNA ID | sgRNA ID | LR738627 |
| SGN001101 | SGN001101 | SGN001819 | SGN001828 |
| SGN000929 | SGN000929 | SGN001820 | SGN001829 |
| SGN000169 | SGN000169 | SGN001821 | SGN001830 |
| SGN000143 | SGN000143 | SGN001018 | SGN001833 |
| SGN000173 | SGN000173 | SGN001822 | SGN001831 |
| SGN000930 | SGN000930 | SGN001823 | SGN001832 |

Cells were then incubated at 37° C. for 72 h. Following incubation, genomic DNA was extracted using NucleoSpin 96 Tissue (Macherey-Nagel) following the manufacturer's protocol. The genomic region flanking the targeted genomic site was PCR amplified using the primers in Table 3 and products were purified using ZR-96 DNA Clean and Concentrator (Zymo Research) following the manufacturer's protocol. The purified PCR products were then sent for Next Generation Sequencing on Illumina MiSeq (2×250). Results were analyzed for INDEL formation and are provided in Table 7.

TABLE 7
Programmable DNA editing with Novel and
Shortened Polymerase III promoters

| | | |
|---|---|---|
| sgRNA ID | sgRNA Target | % Editing |

U6 promoter

| | | |
|---|---|---|
| SGN001101 | SGN001101 | 14.9% |
| SGN000929 | SGN000929 | 15.4% |
| SGN000169 | SGN000169 | 20.1% |
| SGN000143 | SGN000143 | 1.6% |
| SGN000173 | SGN000173 | 14.8% |
| SGN000930 | SGN000930 | 14.9% |

LR738627 promoter

| | | |
|---|---|---|
| SGN001819 | SGN001101 | 10.2% |
| SGN001820 | SGN000929 | 12.2% |
| SGN001821 | SGN000169 | 18.4% |
| SGN001018 | SGN000143 | 0.5% |
| SGN001822 | SGN000173 | 17.2% |
| SGN001823 | SGN000930 | 12.1% |

Mini LR738627 promoter

| | | |
|---|---|---|
| SGN001828 | SGN001101 | 5.6% |
| SGN001829 | SGN000929 | 12.2% |
| SGN001830 | SGN000169 | 18.6% |
| SGN001833 | SGN000143 | 0.7% |
| SGN001831 | SGN000173 | 12.0% |
| SGN001832 | SGN000930 | 11.5% |

Table 7 provides INDEL formation caused by the same sgRNA sequence driven by different polymerase III promoters, demonstrating successful editing at a multitude of targets. The high editing rates confirm that the shortened polymerase III promoters are active for expressing guide RNAs.

That which is claimed:

1. A promoter comprising a nucleotide sequence having at least 90% sequence identity to the nucleotide sequence set forth as any one of SEQ ID NOs: 6-10.

2. The promoter of claim 1, wherein said promoter comprises a nucleotide sequence having 100% sequence identity to the nucleotide sequence set forth as any one of SEQ ID NOs: 6-10.

3. The promoter of claim 1 or 2, wherein said promoter comprises a TATA box sequence.

4. The promoter of any one of claims 1-3, wherein said promoter comprises an octamer (OCT) sequence.

5. The promoter of any one of claims 1-4, wherein said promoter comprises a SPH sequence.

6. The promoter of any one of claims 1-5, wherein said promoter comprises a proximal sequence element (PSE).

7. A vector comprising the promoter of any one of claims 1-6.

8. The vector of claim 7, wherein said vector is a viral vector.

9. The vector of claim 8, wherein said viral vector is an adeno-associated viral (AAV) vector.

10. An expression construct comprising the promoter of any one of claims 1-6 and a coding sequence, wherein said coding sequence is operably linked to said promoter.

11. The expression construct of claim 10, wherein said coding sequence is heterologous to said promoter.

12. The expression construct of claim 10 or 11, wherein said coding sequence encodes an RNA.

13. The expression construct of claim 12, wherein said RNA comprises a guide RNA.

14. The expression construct of claim 10 or 11, wherein said coding sequence encodes a polypeptide.

15. A polynucleotide comprising the expression construct of claim 13 and an RNA-guided nuclease (RGN)-encoding nucleotide sequence, wherein said RGN is capable of binding said guide RNA.

16. The polynucleotide of claim 15, wherein said polynucleotide further comprises an RNA polymerase II (pol II) promoter operably linked to the RGN-encoding nucleotide sequence.

17. The polynucleotide of claim 15 or 16, wherein said RGN is fused to a base-editing polypeptide.

18. The polynucleotide of claim 17, wherein said base-editing polypeptide comprises a cytosine deaminase or adenine deaminase.

19. The polynucleotide of claim 15 or 16, wherein said RGN is fused to a transcriptional activator or transcriptional repressor.

20. The polynucleotide of claim 15 or 16, wherein said RGN is fused to a detectable label.

21. The expression construct of claim 12, wherein said RNA is a crRNA.

22. A polynucleotide comprising the expression construct of claim 21 and a tracrRNA-encoding nucleotide sequence.

23. The polynucleotide of claim 22, wherein said polynucleotide further comprises an RNA polymerase III (pol III) promoter operably linked to said tracrRNA-encoding nucleotide sequence.

24. The polynucleotide of claim 23, wherein said pol III promoter comprises a nucleotide sequence having at least 90% sequence identity to the nucleotide sequence set forth as any one of SEQ ID NOs: 1-10.

25. The polynucleotide of claim 23, wherein said pol III promoter comprises a nucleotide sequence having 100% sequence identity to the nucleotide sequence set forth as any one of SEQ ID NOs: 1-10.

26. The polynucleotide of any one of claims 22-25, wherein said polynucleotide further comprises an RNA-guided nuclease (RGN)-encoding nucleotide sequence, wherein said RGN is capable of binding said crRNA and said tracrRNA.

27. The polynucleotide of claim 26, wherein said polynucleotide further comprises an RNA polymerase II (pol II) promoter operably linked to said RGN-encoding nucleotide sequence.

28. The polynucleotide of claim 26 or 27, wherein said RGN is fused to abase-editing polypeptide.

29. The polynucleotide of claim 28, wherein said base-editing polypeptide comprises a cytosine deaminase or adenine deaminase.

30. The polynucleotide of claim 26 or 27, wherein said RGN is fused to a transcriptional activator or transcriptional repressor.

31. The polynucleotide of claim 26 or 27, wherein said RGN is fused to a detectable label.

32. The expression construct of claim 12, wherein said RNA is a tracrRNA.

33. A polynucleotide comprising the expression construct of claim 32 and a crRNA-encoding nucleotide sequence.

34. The polynucleotide of claim 33, wherein said polynucleotide further comprises an RNA polymerase III (pol III) promoter operably linked to said crRNA-encoding nucleotide sequence.

35. The polynucleotide of claim 34, wherein said pol III promoter comprises a nucleotide sequence having at least 90% sequence identity to the nucleotide sequence set forth as any one of SEQ ID NOs: 1-10.

36. The polynucleotide of claim 34, wherein said pol III promoter comprises a nucleotide sequence having 100% sequence identity to the nucleotide sequence set forth as any one of SEQ ID NOs: 1-10.

37. The polynucleotide of any one of claims 33-36, wherein said polynucleotide further comprises an RNA-guided nuclease (RGN)-encoding nucleotide sequence, wherein said RGN is capable of binding to said crRNA and said tracrRNA.

38. The polynucleotide of claim 37, wherein said polynucleotide further comprises an RNA polymerase II (pol II) promoter operably linked to said RGN-encoding nucleotide sequence.

39. The polynucleotide of claim 37 or 38, wherein said RGN is fused to abase-editing polypeptide.

40. The polynucleotide of claim 39, wherein said base-editing polypeptide comprises a cytosine deaminase or adenine deaminase.

41. The polynucleotide of claim 37 or 38, wherein said RGN is fused to a transcriptional activator or transcriptional repressor.

42. The polynucleotide of claim 37 or 38, wherein said RGN is fused to a detectable label.

43. The expression construct of claim 12, wherein said RNA comprises a gene silencing RNA.

44. A vector comprising the expression construct of any one of claims 10-13, 21, 32, and 43 or the polynucleotide of any one of claims 15-20, 22-31, and 33-42.

45. The vector of claim 44, wherein said vector is a viral vector.

46. The vector of claim 45, wherein said viral vector is an adeno-associated viral (AAV) vector.

47. An expression construct comprising:
a) a promoter comprising a nucleotide sequence having at least 90% sequence identity to the nucleotide sequence set forth as any one of SEQ ID NOs: 1-5; and
b) a heterologous coding sequence operably linked to said promoter.

48. The expression construct of claim 47, wherein said promoter comprises a nucleotide sequence having 100% sequence identity to the nucleotide sequence set forth as any one of SEQ ID NOs: 1-5.

49. The expression construct of claim 47 or 48, wherein said promoter comprises a TATA box sequence.

50. The expression construct of any one of claims 47-49, wherein said promoter comprises an octamer (OCT) sequence.

51. The expression construct of any one of claims 47-50, wherein said promoter comprises a SPH sequence.

52. The expression construct of any one of claims 47-51, wherein said promoter comprises a proximal sequence element (PSE).

53. The expression construct of any one of claims 47-52, wherein said heterologous coding sequence encodes an RNA.

54. The expression construct of claim 53, wherein said RNA comprises a guide RNA.

55. The expression construct of any one of claims 47-52, wherein said heterologous coding sequence encodes a polypeptide.

56. A polynucleotide comprising the expression construct of claim 54 and an RNA-guided nuclease (RGN)-encoding nucleotide sequence, wherein said RGN is capable of binding said guide RNA.

57. The polynucleotide of claim 56, wherein said polynucleotide further comprises an RNA polymerase II (pol II) promoter operably linked to the RGN-encoding nucleotide sequence.

58. The polynucleotide of claim 56 or 57, wherein said RGN is fused to abase-editing polypeptide.

59. The polynucleotide of claim 58, wherein said base-editing polypeptide comprises a cytosine deaminase or adenine deaminase.

60. The polynucleotide of claim 56 or 57, wherein said RGN is fused to a transcriptional activator or transcriptional repressor.

61. The polynucleotide of claim 56 or 57, wherein said RGN is fused to a detectable label.

62. The expression construct of claim 53, wherein said RNA is a crRNA.

63. A polynucleotide comprising the expression construct of claim 62 and a tracrRNA-encoding nucleotide sequence.

64. The polynucleotide of claim 63, wherein said polynucleotide further comprises an RNA polymerase III (pol III) promoter operably linked to said tracrRNA-encoding nucleotide sequence.

65. The polynucleotide of claim 64, wherein said pol III promoter comprises a nucleotide sequence having at least 90% sequence identity to the nucleotide sequence set forth as any one of SEQ ID NOs: 1-10.

66. The polynucleotide of claim 64, wherein said pol III promoter comprises a nucleotide sequence having 100% sequence identity to the nucleotide sequence set forth as any one of SEQ ID NOs: 1-10.

67. The polynucleotide of any one of claims 63-66, wherein said polynucleotide further comprises an RNA-guided nuclease (RGN)-encoding nucleotide sequence, wherein said RGN is capable of binding to said crRNA and said tracrRNA.

68. The polynucleotide of claim 67, wherein said polynucleotide further comprises an RNA polymerase II (pol II) promoter operably linked to said RGN-encoding nucleotide sequence.

69. The polynucleotide of claim 67 or 68, wherein said RGN is fused to abase-editing polypeptide.

70. The polynucleotide of claim 69, wherein said base-editing polypeptide comprises a cytosine deaminase or adenine deaminase.

71. The polynucleotide of claim 67 or 68, wherein said RGN is fused to a transcriptional activator or transcriptional repressor.

72. The polynucleotide of claim 67 or 68, wherein said RGN is fused to a detectable label.

73. The expression construct of claim 53, wherein said RNA is a tracrRNA.

74. A polynucleotide comprising the expression construct of claim 73 and a crRNA-encoding nucleotide sequence.

75. The polynucleotide of claim 74, wherein said polynucleotide further comprises an RNA polymerase III (pol III) promoter operably linked to said crRNA-encoding nucleotide sequence.

76. The polynucleotide of claim 75, wherein said pol III promoter comprises a nucleotide sequence having at least 90% sequence identity to the nucleotide sequence set forth as any one of SEQ ID NOs: 1-10.

77. The polynucleotide of claim 75, wherein said pol III promoter comprises a nucleotide sequence having 100% sequence identity to the nucleotide sequence set forth as any one of SEQ ID NOs: 1-10.

78. The polynucleotide of any one of claims 74-77, wherein said polynucleotide further comprises an RNA-guided nuclease (RGN)-encoding nucleotide sequence, wherein said RGN is capable of binding to said crRNA and said tracrRNA.

79. The polynucleotide of claim 78, wherein said polynucleotide further comprises an RNA polymerase II (pol II) promoter operably linked to said RGN-encoding nucleotide sequence.

80. The polynucleotide of claim 78 or 79, wherein said RGN is fused to abase-editing polypeptide.

81. The polynucleotide of claim 80, wherein said base-editing polypeptide comprises a cytosine deaminase or adenine deaminase.

82. The polynucleotide of claim 78 or 79, wherein said RGN is fused to a transcriptional activator or transcriptional repressor.

83. The polynucleotide of claim 78 or 79, wherein said RGN is fused to a detectable label.

84. The expression construct of claim 57, wherein said RNA comprises a gene silencing RNA.

85. A vector comprising the expression construct of any one of claims 47-55, 62, 73, and 84 or the polynucleotide of any one of claims 56-61, 63-72, and 74-83.

86. The vector of claim 85, wherein said vector is a viral vector.

87. The vector of claim 86, wherein said viral vector is an adeno-associated viral (AAV) vector.

88. A cell comprising the promoter of any one of claims 1-6, the expression construct of any one of claims 10-14, 21, 32, 43, 47-55, 62, 73, and 84, the polynucleotide of any one of claims 15-20, 22-31, 33-42, 56-61, 63-72, and 74-83, or the vector of any one of claims 7-9, 44-46, and 85-87.

89. The cell of claim 88, wherein said cell is a prokaryotic cell.

90. The cell of claim 88, wherein said cell is a eukaryotic cell.

91. The cell of claim 90, wherein said eukaryotic cell is a plant cell.

92. A plant comprising the promoter of any one of claims 1-6, the expression construct of any one of claims 10-14, 21, 32, 43, 47-55, 62, 73, and 84, the polynucleotide of any one of claims 15-20, 22-31, 33-42, 56-61, 63-72, and 74-83, the vector of any one of claims 7-9, 44-46, and 85-87, or the cell of claim 91.

93. A seed comprising the cell of claim 91.

94. A pharmaceutical composition comprising a pharmaceutically acceptable carrier and the expression construct of any one of claims 10-14, 21, 32, 43, 47-55, 62, 73, and 84, the polynucleotide of any one of claims 15-20, 22-31, 33-42, 56-61, 63-72, and 74-83, the vector of any one of claims 44-46 and 85-87, or the cell of claim 90.

95. The pharmaceutical composition of claim 107, wherein the pharmaceutical composition is lipid-based.

96. The pharmaceutical composition of claim 108, wherein the lipid-based pharmaceutical composition comprises liposomes or lipid nanoparticles (LNPs).

97. The pharmaceutical composition of claim 109, wherein the expression construct, the polynucleotide, the vector, or the cell is encapsulated in, and/or non-covalently or covalently attached to the liposomes or the LNPs.

98. A method for preparing an expression construct wherein said method comprises inserting a coding sequence into a nucleic acid molecule comprising the promoter of any one of claims 1-6 such that the coding sequence is operably linked to said promoter.

99. A method for preparing an expression construct wherein said method comprises inserting a promoter comprising a nucleotide sequence having at least 90% sequence identity to the nucleotide sequence set forth as any one of SEQ ID NOs: 6-10 into a nucleic acid molecule comprising a coding sequence such that the coding sequence is operably linked to said promoter.

100. The method of claim 99, wherein said promoter comprises a nucleotide sequence having 100% sequence identity to the nucleotide sequence set forth as any one of SEQ ID NOs: 6-10.

101. The method of any one of claims 98-100, wherein said coding sequence is heterologous to said promoter.

102. The method of any one of claims 98-101, wherein said coding sequence encodes an RNA.

103. The method of claim 102, wherein said RNA comprises a guide RNA.

104. The method of claim 102, wherein said RNA is a crRNA.

105. The method of claim 102, wherein said RNA is a tracrRNA.

106. The method of claim 102, wherein said RNA comprises a gene silencing RNA.

107. The method of any one of claims 98-101, wherein said coding sequence encodes a polypeptide.

108. A method for expressing an RNA, said method comprising contacting in vitro the expression construct of any one of claims 12, 13, 21, 32, 43, 53, 54, 62, 73, and 84, or the polynucleotide of any one of claims 15-20, 22-31, 33-42, 56-61, 63-72, and 74-83 with RNA polymerase III and ribonucleotide triphosphates.

109. A method for expressing an RNA in a cell, said method comprising introducing into said cell the expression construct of any one of claims 12, 13, 21, 32, 43, 53, 54, 62, 73, and 84, or the polynucleotide of any one of claims 15-20, 22-31, 33-42, 56-61, 63-72, and 74-83.

110. A method for expressing a polypeptide in a cell, said method comprising introducing into said cell the expression construct of claim 14.

111. A method for making an RGN ribonucleoprotein complex in a cell, said method comprising introducing into a cell the polynucleotide of any one of claims 15-20, 26-31, 37-42, 56-61, 67-72, and 78-83, wherein the RGN and the gRNA form an RGN ribonucleoprotein complex.

112. The method of claim 111, further comprising purifying said RGN ribonucleoprotein complex.

113. A method for making an mRNA in vitro, said method comprising contacting the expression construct of claim 14 or 55 with RNA polymerase II and ribonucleotide triphosphates.

114. The method of claim 113, wherein said method further comprises contacting the expression construct with a cap analog to generate a capped mRNA.

115. The method of claim 113 or 114, wherein said method further comprises contacting the capped mRNA with a poly(A) polymerase to generate a capped and tailed mRNA.

116. The method of claim 114 or 115, wherein said method further comprises contacting said capped mRNA or said capped and tailed mRNA in vitro with a cell-free lysate to generate an in vitro translated polypeptide.

117. A method for binding a target nucleic acid molecule in a cell, wherein the target nucleic acid molecule comprises a target sequence, said method comprising introducing into said cell the polynucleotide of any one of claims 15-20, 26-31, 37-42, 56-61, 67-72, and 78-83, wherein said guide RNA of any one of claims 15-20, and 56-61 or the crRNA and tracrRNA of the polynucleotide of any one of claims 26-31, 37-42, 67-72, and 78-83 binds to said RGN, thereby directing said RGN to bind to said target sequence within said target nucleic acid molecule.

118. A method for cleaving and/or modifying a target nucleic acid molecule in a cell, wherein said target nucleic acid molecule comprises a target sequence, said method comprising introducing into said cell the polynucleotide of any one of claims 15-18, 26-29, 37-40, 56-59, 67-70, and 78-81, wherein said guide RNA of any one of claims 15-18, and 56-59 or the crRNA and tracrRNA of the polynucleotide of any one of claims 26-29, 37-40, 67-70, and 78-81 binds to said RGN, thereby directing said RGN to bind to said target sequence and cleave and/or modify said target nucleic acid molecule.

119. A method for modifying the expression of a target gene in a cell, wherein said target gene comprises a target sequence, said method comprising introducing into said cell the polynucleotide of any one of claims 19, 30, 41, 60, 71, and 82, wherein said guide RNA of claim 19 or 60 or the crRNA and tracrRNA of the polynucleotide of any one of claims 30, 41, 71, and 82 binds to said RGN, thereby directing said RGN to said target sequence and modifying the expression of said target gene.

120. A method for detecting a target sequence in a cell, said method comprising introducing into said cell the polynucleotide of any one of claims 20, 31, 42, 61, 72, and 83, wherein said guide RNA of claim 20 or 61 or the crRNA and tracrRNA of the polynucleotide of any one of claims 31, 42, 72, and 83 binds to said RGN, thereby directing said RGN to said target sequence; and detecting said detectable label.

121. A method for binding a target nucleic acid molecule in a cell, wherein said target nucleic acid molecule comprises a target sequence, wherein said method comprises introducing into said cell the expression construct of claim 13 or 54, or the polynucleotide of any one of claims 22-25, 33-36, 63-66, and 75-77, wherein said cell comprises an RNA-guided nuclease (RGN); and wherein said guide RNA of claim 13 or 54, or the crRNA and tracrRNA of the polynucleotide of any one of claims 22-25, 33-36, 63-66, and 75-77 binds to said RGN, thereby directing said RGN to bind said target sequence within said target nucleic acid molecule.

122. A method for cleaving and/or modifying a target nucleic acid molecule in a cell, wherein said target nucleic acid molecule comprises a target sequence, wherein said method comprises introducing into said cell the expression construct of claim 13 or 54, or the polynucleotide of any one of claims 22-25, 33-36, 63-66, and 75-77, wherein said cell comprises:
a) an RNA-guided nuclease (RGN); or
b) a fusion protein comprising a nuclease-inactive or nickase RGN fused to a base-editing polypeptide;
and wherein said guide RNA of claim 13 or 54, or the crRNA and tracrRNA of the polynucleotide of any one of claims 22-25, 33-36, 63-66, and 75-77 binds to said RGN, thereby directing said RGN or said fusion protein to bind to said target sequence within said target nucleic acid molecule and cleave and/or modify said target nucleic acid molecule.

123. The method of claim 122, further comprising introducing said RGN or said fusion protein or a nucleic acid molecule encoding the same into said cell prior to or simultaneously with the introduction of said expression construct of claim 13 or 54, or the polynucleotide of any one of claims 22-25, 33-36, 63-66, and 75-77.

124. A method for modifying the expression of a target gene, wherein said target gene comprise a target sequence, wherein said method comprises introducing into said cell the expression construct of claim 13 or 54, or the polynucleotide of any one of claims 22-25, 33-36, 63-66, and 75-77, wherein said cell comprises a fusion protein comprising an RNA-guided nuclease (RGN) fused to a transcriptional activator or transcriptional repressor, and wherein said guide RNA of claim 13 or 54, or the crRNA and tracrRNA of the polynucleotide of any one of claims 22-25, 33-36, 63-66, and 75-77 binds to said RGN, thereby directing said fusion protein to said target sequence and modifying the expression of said target gene.

125. The method of claim 124, further comprising introducing said fusion protein or a nucleic acid molecule encoding the same into said cell prior to or simultaneously with the introduction of said expression construct of claim 13 or 54, or the polynucleotide of any one of claims 22-25, 33-36, 63-66, and 75-77.

126. A method for detecting a target sequence in a cell, wherein said method comprises introducing into said cell the expression construct of claim 13 or 54, or the polynucleotide of any one of claims 22-25, 33-36, 63-66, and 75-77, wherein said cell comprises a fusion protein comprising an RNA-guided nuclease (RGN) fused to a detectable label, and wherein said guide RNA of claim 13 or 54, or the crRNA and tracrRNA of the polynucleotide of any one of claims 22-25, 33-36, 63-66, and 75-77 binds to said RGN, thereby directing said fusion protein to said target sequence; and detecting said detectable label.

127. The method of claim 126, further comprising introducing said fusion protein or a nucleic acid molecule encoding the same into said cell prior to or simultaneously with the introduction of said expression construct of claim 13 or 54, or the polynucleotide of any one of claims 22-25, 33-36, 63-66, and 75-77.

128. A method for binding a target nucleic acid molecule in a cell, wherein said target nucleic acid molecule comprises a target sequence, wherein said method comprises:
a) introducing into said cell the expression construct of claim 13 or 54, or the polynucleotide of any one of claims 22-25, 33-36, 63-66, and 75-77; and
b) introducing into said cell:
i) an RNA-guided nuclease (RGN); or
ii) a nucleic acid molecule encoding an RGN;
wherein said step a) occurs before, during, or after step b); and
wherein said guide RNA of claim 13 or 54, or the crRNA and tracrRNA of the polynucleotide of any one of claims 22-25, 33-36, 63-66, and 75-77 binds to said RGN, thereby directing said RGN to bind said target sequence within said target nucleic acid molecule.

129. A method for cleaving and/or modifying a target nucleic acid molecule in a cell, wherein said target nucleic acid molecule comprises a target sequence, wherein said method comprises:
a) introducing into said cell the expression construct of claim 13 or 54, or the polynucleotide of any one of claims 22-25, 33-36, 63-66, and 75-77; and
b) introducing into said cell:
i) an RNA-guided nuclease (RGN);
ii) a fusion protein comprising a nuclease-inactive or nickase RGN fused to a base-editing polypeptide;
iii) a nucleic acid molecule encoding an RGN; or
iv) a nucleic acid molecule encoding a fusion protein comprising a nuclease-inactive or nickase RGN fused to a base-editing polypeptide;
wherein said step a) occurs before, during, or after step b); and
wherein said guide RNA of claim 13 or 54, or the crRNA and tracrRNA of the polynucleotide of any one of claims 22-25, 33-36, 63-66, and 75-77 binds to said RGN, thereby directing said RGN or said fusion protein to bind to said target sequence and cleave and/or modify said target nucleic acid molecule.

130. The method of claim 129, wherein said step b) iii) comprises introducing a polynucleotide comprising said nucleic acid molecule encoding said RGN or step b) iv) comprises introducing a polynucleotide comprising said nucleic acid molecule encoding said fusion protein, and wherein said polynucleotide further comprises an RNA polymerase II (pol II) promoter operably linked to said nucleic acid molecule encoding said RGN or said nucleic acid molecule encoding said fusion protein.

131. A method for modifying the expression of a target gene in a cell, wherein said target gene comprises a target sequence, wherein said method comprises:
a) introducing into said cell the expression construct of claim 13 or 54, or the polynucleotide of any one of claims 22-25, 33-36, 63-66, and 75-77; and
b) introducing into said cell:
i) a fusion protein comprising a nuclease-inactive RNA-guided nuclease (RGN) fused to a transcriptional activator or transcriptional repressor; or
ii) a nucleic acid molecule encoding a fusion protein comprising a nuclease-inactive RGN fused to a transcriptional activator or transcriptional repressor;
wherein said step a) occurs before, during, or after step b); and
wherein said guide RNA of claim 13 or 54, or the crRNA and tracrRNA of the polynucleotide of any one of claims 22-25, 33-36, 63-66, and 75-77 binds to said RGN, thereby directing said fusion protein to said target sequence and modifying the expression of said target gene.

132. The method of claim 131, wherein said step b) ii) comprises introducing a polynucleotide comprising said nucleic acid molecule encoding said fusion protein, and wherein said polynucleotide further comprises an RNA polymerase II (pol II) promoter operably linked to said nucleic acid molecule encoding said fusion protein.

133. A method for detecting a target sequence in a cell, wherein said method comprises:
a) introducing into said cell the expression construct of claim 13 or 54, or the polynucleotide of any one of claims 22-25, 33-36, 63-66, and 75-77; and
b) introducing into said cell:
i) a fusion protein comprising nuclease-inactive RNA-guided nuclease (RGN) fused to a detectable label; or
ii) a nucleic acid molecule encoding a fusion protein comprising a nuclease-inactive RGN fused to a detection label; and
c) detecting said detectable label;
wherein said step a) occurs before, during, or after step b); and
wherein said guide RNA of claim 13 or 54, or the crRNA and tracrRNA of the polynucleotide of any one of claims 22-25, 33-36, 63-66, and 75-77 binds to said RGN and directs said fusion protein to said target sequence.

134. The method of claim 133, wherein said step b) ii) comprises introducing into said cell a polynucleotide comprising said nucleic acid molecule encoding said fusion protein, and wherein said polynucleotide further comprises an RNA polymerase II (pol II) promoter operably linked to said nucleic acid molecule encoding said fusion protein.

135. A method for reducing the expression of a target gene in a cell, said method comprising introducing the expression construct of claim 43 or 84, wherein said gene silencing RNA reduces the expression of said target gene.

136. A method for treating a disease, said method comprising administering to a subject in need thereof a pharmaceutical composition comprising a pharmaceutically acceptable carrier and a polynucleotide of any one of claims 15-18, 26-29, 37-40, 56-59, 67-70, and 78-81.

137. The method of claim 136, wherein said disease is associated with a causal mutation and said pharmaceutical composition corrects said causal mutation.

138. Use of the polynucleotide of any one of claims 15-18, 26-29, 37-40, 56-59, 67-70, and 78-81 for the manufacture of a medicament useful for treating a disease.

139. The use of claim 138, wherein said disease is associated with a causal mutation and said medicament corrects said causal mutation.

140. A method for treating a disease, said method comprising administering to a subject in need thereof a pharmaceutical composition comprising a pharmaceutically acceptable carrier and a polynucleotide of any one of claims 19, 30, 41, 60, 71, and 82, wherein said disease is associated with the expression of a target gene, wherein said target gene comprises a target sequence, and wherein said guide RNA of said polynucleotide of claim 19 or 60 or the crRNA and tracrRNA of said polynucleotide of any one of claims 30, 41, 71, and 82 binds to said RGN, thereby directing said RGN to said target sequence and modifying the expression of said target gene.

141. Use of the polynucleotide of any one of claims 19, 30, 41, 60, 71, and 82 for the manufacture of a medicament useful for treating a disease associated with the expression of a target gene.

142. A method for treating a disease, said method comprising administering to a subject in need thereof a pharmaceutical composition comprising a pharmaceutically acceptable carrier and an expression construct of claim 43 or 84, wherein said disease is associated with the overexpression of a target gene, and wherein said gene silencing RNA reduces the expression of said target gene.

143. Use of the expression construct of claim 43 or 84 for the manufacture of a medicament useful for treating a disease associated with the overexpression of a target gene.
